(19) United States
(12) Patent Application Publication
(10) Pub. No.: US 2026/0306702 A1
(43) Pub. Date: Oct. 8, 2026

(54) METHODS AND COMPOSITIONS FOR DENTAL CARE

(71) Applicant: NOVABONE PRODUCTS, LLC, Alachua, FL (US)

(72) Inventors: Ashley M. COMPAAN, Alachua, FL (US); Daniel NUNEZ, Alachua, FL (US); Douglas RODRIGUEZ, Alachua, FL (US)

(21) Appl. No.: 19/490,252

(22) PCT Filed: Jun. 5, 2024
(86) PCT No.: PCT/US2024/032603

§ 371 Date: Dec. 4, 2025

Related U.S. Application Data
(60) Provisional application No. 63/506,202, filed on Jun. 5, 2023.

(51) Int. Cl. A61L 26/00

(52) U.S. Cl. CPC A61L 26/0095; A61L 26/0004; A61L 26/0033; A61L 26/0061; A61L 26/0085; A61L 2430/34

(57) ABSTRACT

Disclosed are compositions comprising a tri-layered collagen composition, wherein the composition comprises a first layer comprising a collagen:bioactive glass mixture; a second layer comprising collagen; and a third layer comprising a collagen:bioactive glass mixture. Disclosed are compositions comprising a bilayered collagen composition, wherein the composition comprises a first layer comprising a collagen:bioactive glass mixture and a second layer comprising collagen. Disclosed are methods of restoring bone deficiencies comprising administering one or more of the disclosed compositions to an area needing bone growth. Disclosed are methods of treating a periodontal wound or tissue defect comprising administering one or more of the disclosed compositions to the periodontal wound or tissue defect. Disclosed are methods of making the disclosed compositions.

### CROSS-REFERENCE TO RELATED APPLICATIONS

This application claims the benefit of U.S. Application No. 63/506,202, filed on Jun. 5, 2023, the contents of which are incorporated herein by reference in their entirety.

### BACKGROUND

Promising procedures for bone reconstruction in dentistry are guided tissue regeneration (GTR) and guided bone regeneration (GBR). Both of these surgical methods involve the restoration of bone deficiencies by means of barrier membranes. GTR implies the regeneration of the bone and attachment anatomical structures (periodontal ligament, cementum) of living teeth, whereas GBR includes the placement of a membrane into the area where the new formation of bone is intended. For either technique, bone or substitute material is used in combination with a barrier membrane. Barrier membranes are critical for the restoration of the lost tissues. Thus, there is a need for new and improved barrier membranes to be used in dentistry.

Wound healing in the oral cavity is typically characterized by healing of the palate and gingival tissue in the presence of healthy underlying bones and without scar tissue formation. This is due to early onset of the inflammatory phase, decreased levels of immunity mediators, fewer blood vessels, more cells originating from the bone marrow, rapid re-epithelialization, and rapid fibroblast proliferation. Wound healing in the palate is more difficult in the absence of healthy underlying bone. In such cases, wound healing might be accompanied by perforation to the nose and antrum or by serious scarring. This can lead to narrowing of the transversal width of the maxilla if the patient is in their growing phase, as is observed in cleft patients who have surgery of the palate.

Wound healing after periodontal surgery carries a notable risk of degradation of the interdental papilla, with the occurrence of black triangles. Specific incisions and flaps are important to ensure minimum retraction, and good oral hygiene is critical to avoid inflammation.

A specific form of wound healing occurs around dental implants. In biphasic dental implant procedures, the implant is placed directly under or at the same level as the bone surface. The cover screw is overlaid with soft tissues that heal without substantial granulation tissue formation. Healing in the bone occurs between the edge surface of the implant and the prepared osteotomy edge. Blood clots form mainly at the inner side of the implant grooves, and are then infiltrated by granulocytes and macrophages. Fibroblastic progenitor cells migrate into the provisional matrix, enabling formation of granulation tissue, which is then vascularized by endothelial cell migration. Finally, the cells in the granulation tissue differentiate into osteoblasts, creating bone.

Current clinical results are generally limited to ineffective tissue repair leading to a continued chronic open wound, infection and/or scarring.

There is a continued need for a safe and effective dental wound care treatments.

### BRIEF SUMMARY

Disclosed are compositions comprising a tri-layered collagen composition, wherein the composition comprises a first layer comprising a collagen:bioactive glass mixture; a second layer comprising collagen; and a third layer comprising a collagen:bioactive glass mixture.

Disclosed are compositions comprising a bilayered collagen composition, wherein the composition comprises a first layer comprising a collagen:bioactive glass mixture and a second layer comprising collagen.

Disclosed are methods of restoring bone deficiencies comprising administering one or more of the disclosed compositions as a barrier membrane in guided tissue regeneration or guided bone regeneration.

Disclosed are methods of managing a periodontal wound or soft tissue defect comprising administering one or more of the disclosed compositions to the periodontal wound or soft tissue defect.

Disclosed are methods of treating a periodontal wound or tissue defect comprising administering one or more of the disclosed compositions to the periodontal wound or tissue defect.

Disclosed are methods of making a tri-layer collagen composition comprising preparing a collagen slurry; adding bioactive glass to the collagen slurry, forming a bioactive glass collagen slurry mix; coating the first side of a collagen base, having a first side and a second side, with the bioactive glass collagen slurry mix, forming a single-coated collagen base; drying the single-coated collagen base: coating the second side of the collagen base with the bioactive glass collagen slurry mix, forming a double-coated collagen base; drying the double-coated collagen base, thereby forming a dried collagen product; pressing the dried collagen product, thereby forming a pressed, dried collagen product; drying the pressed, lyophilized collagen product: thereby forming a dried, pressed collagen product; crosslinking the dried collagen product, thereby forming a crosslinked collagen product; drying the crosslinked collagen product, thereby forming a dried, crosslinked collagen product; pressing the dried, crosslinked collagen product, thereby forming a pressed, dried, crosslinked collagen product; and drying the pressed, dried, crosslinked collagen product, thereby forming a tri-layered collagen product.

Disclosed are methods of making a tri-layer collagen composition comprising preparing a collagen slurry; perforating the first side of a collagen base, having a first side and a second side, thereby forming a perforated collagen base; adding bioactive glass to the first side of the perforated collagen base; coating the first side of the perforated collagen base comprising bioactive glass with the collagen slurry, thereby forming a single-coated collagen base; drying the single-coated collagen base: perforating the second side of the collagen base; adding bioactive glass to the second side of the perforated collagen base; coating the second side of the perforated collagen base comprising bioactive glass with the collagen slurry, thereby forming a double-coated collagen base; lyophilizing the double-coated collagen base, thereby forming a lyophilized collagen product; pressing the lyophilized collagen product, thereby forming a pressed, lyophilized collagen product; drying the pressed, lyophilized collagen product; forming a dried collagen product; crosslinking the dried collagen product, thereby forming a crosslinked collagen product; drying the crosslinked collagen product, thereby forming a dried, crosslinked collagen product; pressing the dried, crosslinked collagen product, thereby forming a pressed, dried, crosslinked collagen product; drying the pressed, dried, crosslinked collagen product, thereby forming a tri-layered collagen product.

Disclosed are methods of making a tri-layer collagen composition comprising preparing a collagen slurry; lyophilizing at least a portion of the collagen slurry; pressing the lyophilized collagen slurry, thereby forming a collagen base; adding bioactive glass to a collagen slurry, thereby forming a bioactive glass collagen slurry mix; coating the first side of the collagen base, having a first side and a second side, with the bioactive glass collagen slurry mix, thereby forming a single-coated collagen base; drying the single-coated collagen base; coating the second side of the single-coated collagen base with the bioactive glass collagen slurry mix, thereby forming a double-coated collagen base; lyophilizing the double-coated collagen base, thereby forming a lyophilized collagen product: pressing the lyophilized collagen product, thereby forming a pressed, lyophilized collagen product; drying the pressed, lyophilized collagen product; thereby forming a dried collagen product; crosslinking the dried collagen product, thereby forming a crosslinked collagen product; drying the crosslinked collagen product, thereby forming a dried, crosslinked collagen product: pressing the dried, crosslinked collagen product, thereby forming a pressed, dried, crosslinked collagen product; and drying the pressed, dried, crosslinked collagen product, thereby forming a tri-layered collagen product.

Disclosed are methods of making a tri-layer collagen composition comprising preparing a collagen slurry; lyophilizing at least a portion of the collagen slurry; pressing the lyophilized collagen slurry to form a collagen base; perforating the first side of a collagen base, having a first side and a second side, thereby forming a perforated collagen base; adding bioactive glass to the first side of the perforated collagen base: coating the first side of the perforated collagen base comprising bioactive glass with the collagen slurry, thereby forming a single-coated collagen base; drying the single-coated collagen base; perforating the second side of the collagen base; adding bioactive glass to the second side of the perforated collagen base; coating the second side of the perforated collagen base comprising bioactive glass with the collagen slurry, thereby forming a double-coated collagen base: lyophilizing the double-coated collagen base, thereby forming a lyophilized collagen product; pressing the lyophilized collagen product, thereby forming a pressed, lyophilized collagen product; drying the pressed, lyophilized collagen product, thereby forming a dried collagen product; crosslinking the dried collagen product; drying the crosslinked collagen product, thereby forming a dried, crosslinked collagen product; pressing the dried, crosslinked collagen product, thereby forming a pressed, dried, crosslinked collagen product: drying the pressed, dried, crosslinked collagen product, thereby forming a tri-layered collagen product.

Disclosed are methods of making a bilayer collagen composition comprising preparing a collagen slurry; adding bioactive glass to the collagen slurry, forming a bioactive glass collagen slurry mix; coating one side of a collagen base with the bioactive glass collagen slurry mix, forming a single-coated collagen base; drying the single-coated collagen base, thereby forming a dried collagen product; pressing the dried collagen product, thereby forming a pressed, dried collagen product; drying the pressed, dried collagen product, thereby forming a dried collagen product; crosslinking the dried collagen product, thereby forming a crosslinked collagen product; drying the crosslinked collagen product, thereby forming a dried, crosslinked collagen product; pressing the dried, crosslinked collagen product, thereby forming a pressed, dried, crosslinked collagen product; drying the pressed, dried, crosslinked collagen product, thereby forming a bilayered collagen product.

Disclosed are methods of making a bilayer collagen composition comprising preparing a collagen slurry; lyophilizing at least a portion of the collagen slurry; pressing the lyophilized collagen slurry to form a collagen base; perforating one side of a collagen base; adding bioactive glass to the perforated side of the collagen base; coating the perforated collagen base side comprising bioactive glass with the collagen slurry, thereby forming a single-coated collagen base; drying the single-coated collagen base: thereby forming a dried, collagen product; pressing the dried collagen product, thereby forming a pressed, dried collagen product; drying the pressed, dried collagen product, thereby forming a dried collagen product; crosslinking the dried collagen product, thereby forming a crosslinked collagen product; drying the crosslinked collagen product, thereby forming a dried, crosslinked collagen product; pressing the dried, crosslinked collagen product, thereby forming a pressed, dried, crosslinked collagen product: drying the pressed, dried, crosslinked collagen product, thereby forming a tri-layered collagen product.

Disclosed are methods of making a bilayer collagen composition comprising preparing a collagen slurry; lyophilize at least a portion of the collagen slurry; pressing the lyophilized collagen slurry to form a collagen base; adding bioactive glass to a collagen slurry, forming a bioactive glass collagen slurry mix; coating one side of the collagen base with the bioactive glass collagen slurry mix, thereby forming a single-coated collagen base; drying the single-coated collagen base; lyophilizing the single-coated collagen base, thereby forming a lyophilized collagen product; pressing the lyophilized collagen product, thereby forming a pressed, lyophilized collagen product; drying the pressed, lyophilized collagen product, thereby forming a dried collagen product: crosslinking the dried collagen product, thereby forming a crosslinked collagen product; drying the crosslinked collagen product, thereby forming a dried, crosslinked collagen product: pressing the dried, crosslinked collagen product, thereby forming a pressed, dried, crosslinked collagen product; and drying the pressed, dried, crosslinked collagen product, thereby forming a bilayered collagen product.

Disclosed are methods of making a bilayer collagen composition comprising preparing a collagen slurry; lyophilizing at least a portion of the collagen slurry; pressing the lyophilized collagen slurry to form a collagen base; perforating one side of a collagen base; adding bioactive glass to the perforated collagen base; coating the side of the perforated collagen base comprising bioactive glass with the collagen slurry, forming a single-coated collagen base; drying the single-coated collagen base; lyophilizing the single-coated collagen base, thereby forming a lyophilized collagen product; pressing the lyophilized collagen product, thereby forming a pressed, lyophilized collagen product; drying the pressed, lyophilized collagen product, thereby forming a dried collagen product; crosslinking the dried collagen product, thereby forming a crosslinked collagen product; drying the crosslinked collagen product, thereby forming a dried, crosslinked collagen product; pressing the dried, crosslinked collagen product, thereby forming a pressed, dried, crosslinked collagen product; drying the pressed, dried, crosslinked collagen product, thereby forming a bilayered collagen product.

Disclosed are methods of making a composition comprising preparing a collagen slurry; lyophilizing the collagen slurry to create a lyophilized collagen sample; cross-linking the collagen slurry before and/or after lyophilization; adding bioactive glass to a cross-linked lyophilized collagen sample.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a trilayer composition with bioactive glass in the collagen slurry layers.

FIG. 2 shows a bilayer composition with bioactive glass in the collagen slurry layer.

FIG. 3 shows a trilayer composition with bioactive glass in the collagen base.

FIG. 4 shows a bilayer composition with bioactive glass in the collagen base.

FIG. 5 shows a trilayer composition with bioactive glass in the collagen slurry and a lyophilized collagen base.

FIG. 6 shows a bilayer composition with bioactive glass in the collagen slurry and a lyophilized collagen base.

FIG. 7 shows a trilayer composition with bioactive glass in the lyophilized collagen base.

FIG. 8 shows a bilayer composition with bioactive glass in the lyophilized collagen base.

FIG. 9 shows a monolayer composition with bioactive glass in the collagen slurry.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C: D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a composition” includes a plurality of such compositions, reference to “the collagen” is a reference to one or more collagens and equivalents thereof known to those skilled in the art, and so forth.

As used herein, the term “subject” or “patient” can be used interchangeably and refer to any organism to which a composition of the invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates, and the like.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Compositions

Disclosed are compositions comprising collagen and bioactive glass. In some aspects, the compositions can be in a sheet-like configuration.

### 1. Trilayer Composition

Disclosed are compositions comprising a trilayered collagen composition, wherein the composition comprises a first layer comprising a collagen:bioactive glass mixture; a second layer comprising collagen; and a third layer comprising a collagen:bioactive glass mixture. Disclosed are compositions comprising a trilayered collagen composition, wherein the composition comprises a first layer comprising a collagen slurry mixture; a second layer comprising perforated collagen, wherein bioactive glass is present in the perforations; and a third layer comprising a collagen slurry mixture.

In some aspects, the disclosed compositions can be lyophilized or freeze-dried.

In some aspects, the disclosed compositions have a density of about 300-1000 mg/cc. In some aspects, the disclosed compositions have a density of about 600 mg/cc.

In some aspects, the disclosed compositions have a pH of about 7-10. In some aspects, the disclosed compositions have a pH of about 8-10. In some aspects, the higher pH can be due to the basic bioactive glass despite the acidic collagen matrix.

In some aspects, the disclosed compositions have a thickness of about 0.10 mm-0.45 mm.

Disclosed are compositions comprising about 60-95%/wt collagen and about 1-40% bioactive glass, wherein the collagen is cross-linked. Disclosed are compositions comprising about 70-99%/wt collagen and about 1-30% bioactive glass, wherein the collagen is crosslinked. Disclosed are compositions comprising about 3-60%/wt collagen and about 40-97% bioactive glass, wherein the collagen is crosslinked. In some aspects, the composition comprises 1% salts. In some aspects, the composition comprises ≤1% water.

i. Collagen

Disclosed are compositions comprising collagen and bioactive glass. In some aspects, collagen is present in all three layers.

In some aspects, the collagen in all three layers is derived from the same source. In some aspects, the collagen in the first and third layers is derived from the same source. In some aspects, the collagen in all three layers is derived from different sources.

In some aspects, the collagen is marine (e.g. fish), bovine, porcine, ovine, vegan, equine, mammalian, jellyfish, avian, reptilian, amphibian, bioengineered, or recombinant collagen. In some aspects, the collagen can be from any area of a human, cow, pig, sheep, rodent, or horse. For example, the collagen can be skin-derived, tendon-derived or hide-derived.

In some aspects, the collagen is purified collagen, wherein it has been purified from any of the sources described herein. In some aspects, the collagen has negligible amounts of non-collagen components derived from the collagen source. For example, the collagen has negligible amounts of cells, fat, growth factors, or other proteins. In some aspects, the collagen is purified type I collagen derived from bovine flexor tendon.

In some aspects, the collagen is synthetic or recombinant. In some aspects, synthetic collagen retains the structural characteristics. In some aspects, synthetic or recombinant collagen can be any material that forms a resorbable sponge.

In some aspects, the collagen of the disclosed compositions is crosslinked. In some aspects, the collagen of only the middle layer is crosslinked. In some aspects, the collagen of all three layers is crosslinked. In some aspects, only one of the collagen layers is crosslinked. In some aspects, at least two of the collagen layers are crosslinked.

In some aspects, the compositions comprise 70-99% collagen. In some aspects, the compositions comprise 82-88% collagen. In some aspects, each of the three layers comprises the same amount of collagen. In some aspects, the first layer and the third layer of the composition comprise the same amount of collagen. In some aspects, because the second layer does not comprise bioactive glass, the second layer comprises about ≥97%/wt collagen.

In some aspects, the collagen base can be perforated on one or both sides. In some aspects, the perforations can be from a pinned roller tool. In some aspects, the perforations do not extend all the way through the collagen base. In some aspects, the perforated collagen base comprises bioactive glass. For example, in some aspects, the bioactive glass is present in the perforations.

In some aspects, the collagen base can be a lyophilized collagen slurry which is a sponge-like material. Instead of being pure collagen, in some aspects, the pure collagen is made into a collagen slurry (e.g. homogenization) and then lyophilized.

In some aspects, the collagen base comprises pores. In some aspects, the pores allow for bodily fluids to be absorbed into the collagen base and enable vascularization.

ii. Crosslinking Agent

In some aspects, the disclosed compositions comprise a crosslinking agent. In some aspects, the crosslinking agent is citric acid, glycine, lysine, glutamic acid, serine, trilysine, glutaraldehyde, transglutaminase, ethylene oxide, phosphate (cross linking aid), tris, carbodiimide, heat, irradiation, EDC-NHS, ribose and other sugars, or combinations thereof.

In some aspects, the compositions comprise 0-3% crosslinking agent. In some aspects, the compositions comprise <10% crosslinking agent. In some aspects, the cross-linking agent is glutaraldehyde, wherein the glutaraldehyde is in a final concentration of about 0-3%. In some aspects, the cross-linking agent is glutaraldehyde, wherein the glutaraldehyde is in a final concentration of <1%. In some aspects, the compositions can be crosslinked in a 0.05% glutaraldehyde solution.

In some aspects, disclosed are compositions comprising collagen and bioactive glass, wherein the collagen is crosslinked but no crosslinking agent is present. For example, crosslinking of the collagen can occur using physicothermal methods, including, but not limited to, UV cross-linking, and therefore no crosslinking agent is present in the composition.

In some aspects, the crosslinking agent is only in the middle layer of the composition. In some aspects, the crosslinking agent is only in one layer of the composition. In some aspects, the crosslinking agent is in at least two layers of the composition. In some aspects, the crosslinking agent is in all three layers of the composition.

iii. Bioactive Glass

In some aspects, the disclosed compositions can comprise bioactive glass. In some aspects, the bioactive glass is in the first layer and third layer of the compositions. Thus, in some aspects, the bioactive glass is not present in the second layer (middle layer). In some aspects, the bioactive glass is in at least one layer of the compositions. In some aspects, the bioactive glass is only present in the second layer (i.e., perforated collagen base).

In some aspects, the bioactive glass is 45S5, 58S, S53P4, or a Borate-formulation. In some aspects, the bioactive glass comprises 12-45% CaO, 0-65% SiO2, 0-30% Na2O, 2-17% P2O5, 0-7% MgO, 0-1% CaF2, 0-60% B2O3, and 0-15% K2O by mass percent.

In some aspects, the bioactive glass can be formed via melt or sol-gel glass processing methods. In some aspects, sol-gel derived glass can have dense or porous morphologies. In some aspects, porous morphology results in higher surface area, leading to faster rates of dissolution.

In some aspects, the bioactive glass has a particle size of about 10-1000 m. For example, the composition can have a mean particle size of about 50. In some aspects, the bioactive glass is in the form of particles having a particle size of about 1-600 μm.

In some aspects, the disclosed compositions comprise a non-porous bioactive glass. In some aspects, the disclosed composition comprise a porous bioactive glass. In some aspects, the bioactive glass has a porosity of up to 90%. In some aspects, the bioactive glass has pores ranging from about 1 to about 1000 microns. In some aspects, the bioactive glass has an average pore size of 100 microns plus or minus 50 microns, 200 microns plus or minus 50 microns, 300 microns plus or minus 50 microns, 400 microns plus or minus 50 microns, 500 microns plus or minus 50 microns, 600 microns plus or minus 50 microns, 700 microns plus or minus 50 microns.

In some aspects, the bioactive glass is in the form of irregular granules. In some aspects, the bioactive glass is in the form of approximately spherical particles. In some aspects, the bioactive glass is in the form of fibers.

In some aspects, a smaller size range of bioactive glass can be used for a homogenously dispersed monolayer composition. In some aspects, the bioactive glass is naturally buoyant and therefore no settling occurs via gravity.

In some aspects, a larger size range of bioactive glass can be used for a composition that is functionally graded through-the-thickness. In some aspects, larger bioactive glass can settle near the bottom of the composition during manufacturing.

In some aspects, a combination of smaller and larger size ranges of bioactive glass are present in the disclosed compositions, with smaller particles dispersed throughout the composition and large particles that are pre-dominantly on one side due to settling.

In some aspects, the bioactive glass is distributed uniformly throughout the collagen. In some aspects, the bioactive glass is distributed unevenly, or randomly, throughout the collagen. In some aspects, at least one layer of the compositions comprise bioactive glass that is distributed uniformly throughout the collagen. In some aspects, at least two layers of the compositions comprise bioactive glass that is distributed uniformly throughout the collagen. In some aspects, the bioactive glass is distributed uniformly throughout the collagen in the first layer and the third layer.

In some aspects, the compositions comprise 1-30% bioactive glass. In some aspects, the compositions comprise 12-18% bioactive glass.

In some aspects, the bioactive glass described throughout can be substituted for one or more of the following: Beta-tricalcium phosphate (β-TCP), hydroxyapatite (HA), carbonated hydroxyapatite, calcium sulfate, calcium phosphate, calcium apatite, aragonite, or any mixture of these with each other or with bioactive glass. Thus, any of disclosed compositions or methods of making or using the disclosed compositions can have any of these substitutes instead of bioactive glass. For example, in some aspects, the composition 1-30% β-TCP. In some aspects, the compositions comprise 12-18% β-TCP. In some aspects, the compositions comprise about 60-95%/wt collagen and about 1-40% β-TCP, wherein the collagen is cross-linked. Disclosed are compositions comprising about 70-99%/wt collagen and about 1-30% β-TCP, wherein the collagen is crosslinked. Disclosed are compositions comprising about 3-60%/wt collagen and about 40-97% β-TCP, wherein the collagen is crosslinked.

### 2. Bilayer Compositions

Disclosed are compositions comprising a bilayered collagen composition, wherein the composition comprises a first layer comprising a collagen:bioactive glass mixture and a second layer comprising collagen. Disclosed are compositions comprising a bilayered collagen composition, wherein the composition comprises a first layer comprising a collagen slurry mixture and a second layer comprising perforated collagen, wherein bioactive glass is present in the perforations.

The bilayer compositions can be identical to the disclosed trilayer collagen compositions except without the third layer.

In some aspects, the disclosed compositions can be lyophilized or freeze-dried.

In some aspects, the disclosed compositions have a density of about 300-1000 mg/cc. In some aspects, the disclosed compositions have a density of about 600 mg/cc.

In some aspects, the disclosed compositions have a pH of about 7-10. In some aspects, the disclosed compositions have a pH of about 8-10. In some aspects, the higher pH can be due to the basic bioactive glass despite the acidic collagen matrix.

In some aspects, the disclosed compositions have a thickness of about 0.10 mm-0.45 mm.

Disclosed are compositions comprising about 60-95%/wt collagen and about 1-40% bioactive glass, wherein the collagen is crosslinked. Disclosed are compositions comprising about 70-99%/wt collagen and about 1-30% bioactive glass, wherein the collagen is crosslinked. Disclosed are compositions comprising about 3-60%/wt collagen and about 40-97% bioactive glass, wherein the collagen is crosslinked. In some aspects, the composition comprises ≤1% salts.

i. Collagen

Disclosed are compositions comprising collagen and bioactive glass. In some aspects, collagen is present in both layers.

In some aspects, the collagen in both layers is derived from the same source. In some aspects, the collagen in each layer is derived from different sources.

In some aspects, the collagen is marine (e.g. fish), bovine, porcine, ovine, vegan, equine, mammalian, jellyfish, avian, reptilian, amphibian, bioengineered, or recombinant collagen. In some aspects, the collagen can be from any area of a human, cow, pig, sheep, rodent, or horse. For example, the collagen can be skin-derived, tendon-derived or hide-derived.

In some aspects, the collagen is purified collagen, wherein it has been purified from any of the sources described herein. In some aspects, the collagen has negligible amounts of non-collagen components derived from the collagen source. For example, the collagen has negligible amounts of cells, fat, growth factors, or other proteins. In some aspects, the collagen is purified type I collagen derived from bovine flexor tendon.

In some aspects, the collagen is synthetic or recombinant. In some aspects, synthetic collagen retains the structural characteristics. In some aspects, synthetic or recombinant collagen can be any material that forms a resorbable sponge.

In some aspects, the collagen of the disclosed compositions is crosslinked. In some aspects, the collagen of only the second layer is crosslinked. In some aspects, the collagen of both layers is crosslinked.

In some aspects, the compositions comprise 70-99% collagen. In some aspects, the compositions comprise 82-88% collagen. In some aspects, each of the layers comprises the same amount of collagen. In some aspects, the second layer of the composition comprise a higher amount of collagen. In some aspects, because the second layer does not comprise bioactive glass, the second layer comprises about ≥97%/wt collagen.

In some aspects, the collagen base can be perforated on at least one side. In some aspects, the collagen base is perforated on the side that is coated with a collagen slurry. In some aspects, the perforations can be from a pinned roller tool. In some aspects, the perforations do not extend all the way through the collagen base. In some aspects, the perforated collagen base comprises bioactive glass. For example, in some aspects, the bioactive glass is present in the perforations.

In some aspects, the collagen base can be a lyophilized collagen slurry which is a sponge-like material. Instead of being pure collagen, in some aspects, the pure collagen is made into a collagen slurry (e.g. homogenization) and then lyophilized.

In some aspects, the collagen base comprises pores. In some aspects, the pores allow for bodily fluids to be absorbed into the collagen base and enable vascularization.

ii. Crosslinking Agent

In some aspects, the disclosed compositions comprise a crosslinking agent. In some aspects, the crosslinking agent is citric acid, glycine, lysine, glutamic acid, serine, trilysine, glutaraldehyde, transglutaminase, ethylene oxide, phosphate (cross linking aid), tris, carbodiimide, heat, irradiation. EDC-NHS, ribose and other sugars, or combinations thereof.

In some aspects, the compositions comprise 0-3% crosslinking agent. In some aspects, the compositions comprise <10% crosslinking agent. In some aspects, the crosslinking agent is glutaraldehyde, wherein the glutaraldehyde is in a final concentration of about 0-3%. In some aspects, the crosslinking agent is glutaraldehyde, wherein the glutaraldehyde is in a final concentration of <1%. In some aspects, the compositions can be crosslinked in a 0.05% glutaraldehyde solution.

In some aspects, disclosed are compositions comprising collagen and bioactive glass, wherein the collagen is crosslinked but no crosslinking agent is present. For example, crosslinking of the collagen can occur using physicothermal methods, including, but not limited to, UV cross-linking, and therefore no crosslinking agent is present in the composition.

In some aspects, the crosslinking agent is only in the second layer of the composition. In some aspects, the crosslinking agent is only in one layer of the composition. In some aspects, the crosslinking agent is in both layers of the composition.

iii. Bioactive Glass

In some aspects, the disclosed compositions can comprise bioactive glass. In some aspects, the bioactive glass is in the first layer of the compositions only. Thus, in some aspects, the bioactive glass is not present in the second layer. In some aspects, the bioactive glass is only present in the second layer (i.e., perforated collagen base).

In some aspects, the bioactive glass is 45S5, 58S, S53P4, or a Borate-formulation. In some aspects, the bioactive glass comprises 12-45% CaO, 0-65% SiO2, 0-30% Na2O, 2-17% P2O5, 0-7% MgO, 0-1% CaF2, 0-60% B2O3, and 0-15% K2O by mass percent.

In some aspects, the bioactive glass can be formed via melt or sol-gel glass processing methods. In some aspects, sol-gel derived glass can have dense or porous morphologies. In some aspects, porous morphology results in higher surface area, leading to faster rates of dissolution.

In some aspects, the bioactive glass has a particle size of about 10-1000 μm. For example, the composition can have a mean particle size of about 50. In some aspects, the bioactive glass is in the form of particles having a particle size of about 1-600 μm.

In some aspects, the disclosed compositions comprise a non-porous bioactive glass. In some aspects, the disclosed composition comprise a porous bioactive glass. In some aspects, the bioactive glass has a porosity of up to 90%. In some aspects, the bioactive glass has pores ranging from about 1 to about 1000 microns. In some aspects, the bioactive glass has an average pore size of 100 microns plus or minus 50 microns, 200 microns plus or minus 50 microns, 300 microns plus or minus 50 microns, 400 microns plus or minus 50 microns, 500 microns plus or minus 50 microns, 600 microns plus or minus 50 microns, 700 microns plus or minus 50 microns.

In some aspects, the bioactive glass is in the form of irregular granules. In some aspects, the bioactive glass is in the form of approximately spherical particles. In some aspects, the bioactive glass is in the form of fibers.

In some aspects, a smaller size range of bioactive glass can be used for a homogenously dispersed monolayer composition. In some aspects, the bioactive glass is naturally buoyant and therefore no settling occurs via gravity.

In some aspects, a larger size range of bioactive glass can be used for a composition that is functionally graded through-the-thickness. In some aspects, larger bioactive glass can settle near the bottom of the composition.

In some aspects, a combination of smaller and larger size ranges of bioactive glass are present in the disclosed compositions, with smaller particles dispersed throughout the composition and large particles that are pre-dominantly on one side due to settling.

In some aspects, the bioactive glass is distributed uniformly throughout the collagen. In some aspects, the bioactive glass is distributed unevenly, or randomly, throughout the collagen. In some aspects, at least one layer of the compositions comprise bioactive glass that is distributed uniformly throughout the collagen. In some aspects, both layers of the compositions comprise bioactive glass that is distributed uniformly throughout the collagen. In some aspects, the bioactive glass is distributed uniformly throughout the collagen in the first layer layer.

In some aspects, the compositions comprise 1-30% bioactive glass. In some aspects, the compositions comprise 12-18% bioactive glass.

As described for the trilayer compositions, the bilayer (and monolayer) compositions can have similar alternatives as well. In some aspects, the bioactive glass described throughout can be substituted for one or more of the following: Beta-tricalcium phosphate (β-TCP), hydroxyapatite (HA), carbonated hydroxyapatite, calcium sulfate, calcium phosphate, calcium apatite, aragonite, or any mixture of these with each other or with bioactive glass. Thus, any of disclosed compositions or methods of making or using the disclosed compositions can have any of these substitutes instead of bioactive glass. For example, in some aspects, the composition 1-30% β-TCP. In some aspects, the compositions comprise 12-18% β-TCP. In some aspects, the compositions comprise about 60-95%/wt collagen and about 1-40% β-TCP, wherein the collagen is cross-linked. Disclosed are compositions comprising about 70-99%/wt collagen and about 1-30% β-TCP, wherein the collagen is crosslinked. Disclosed are compositions comprising about 3-60%/wt collagen and about 40-97% β-TCP, wherein the collagen is crosslinked.

### C. Methods of Bone or Tissue Regeneration

In periodontal disease, bone breaks down. Treatment for periodontal disease can be the process of rebuilding the bone and/or tissue. In some aspects, the disclosed compositions can act as a barrier membrane and prevent epithelium, which regenerates relatively quickly, from growing into an area in which another, more slowly growing tissue type, such as bone, is desired.

Disclosed are methods of restoring bone deficiencies comprising administering one or more of the disclosed compositions to a subject in need thereof.

Disclosed are methods of stimulating or guiding bone growth comprising administering one or more of the disclosed compositions to a subject in need thereof.

In some aspects, the compositions act as a barrier membrane in guided tissue regeneration (GTR) or guided bone regeneration (GBR).

In some aspects, GTR or GBR can be used to periodontal diseases or treatments. For example, a periodontal treatment can be selected from the group consisting of implants, ridge reconstruction, bone defects, mucogingival flap surgery, frenectomy, or combinations thereof.

In some aspects, any of the disclosed compositions can be used in the disclosed methods. For example, the disclosed trilayer composition, bilayer composition or monolayer composition can be used.

In some aspects, the disclosed compositions are applied directly to the area in need of bone regeneration/stimulation.

### D. Methods of Wound Healing

Disclosed are methods of managing a periodontal wound or soft tissue defect comprising administering one or more of the disclosed compositions to the periodontal wound or soft tissue defect. In some aspects, managing a periodontal wound or soft tissue defect comprises repairing or treating the periodontal wound or soft tissue defect.

Disclosed are methods of treating a periodontal wound or tissue defect comprising administering one or more of the disclosed compositions to the periodontal wound or tissue defect. In some aspects, the disclosed compositions can be used during periodontal procedures.

For example, a periodontal wound can be selected from the group consisting of a wound that results from microbial infection, gingivitis, periodontitis, mechanical injury, or periodontal surgery. Examples of periodontal wounds include, but are not limited to wounds in the epithelium, gingival alveolar mucosa, periodontal connective tissue, bone, root cementum as well as loss of tissue and bony defects and combinations thereof. In some aspects, a periodontal wound or tissue defect can be in connection with a periodontal surgery. In some aspects, periodontal surgery is a form of dental surgery that prevents or corrects anatomical, traumatic, developmental, or plaque-induced defects in the bone, gingiva, or alveolar mucosa. For example, a periodontal surgery or procedure can be selected from the group consisting of implants, ridge reconstruction, bone defects, mucogingival flap surgery, frenectomy, or combinations thereof.

In some aspects, administering can mean placing the composition on or around the periodontal wound or tissue defect.

### E. Methods of Making

Disclosed are methods of making collagen compositions comprising collagen and bioactive glass.

### 1. Trilayer

Disclosed are methods of making a tri-layer collagen composition comprising preparing a collagen slurry; adding bioactive glass, coating a first and second side of a collagen base with the bioactive glass collagen slurry mix, lyophilizing, pressing, and crosslinking. Examples of each of these steps is described in more detail below and the order of each of these steps can be changed. For example, cross-linking can be performed before or after any of the drying steps or the pressing step.

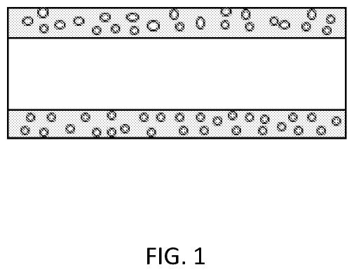

Disclosed are methods of making a tri-layer collagen composition comprising preparing a collagen slurry; adding bioactive glass to the collagen slurry, forming a bioactive glass collagen slurry mix; coating the first side of a collagen base, having a first side and a second side, with the bioactive glass collagen slurry mix, forming a single-coated collagen base; drying the single-coated collagen base; coating the second side of the collagen base with the bioactive glass collagen slurry mix, forming a double-coated collagen base; drying the double-coated collagen base, thereby forming a dried collagen product; pressing the dried collagen product, thereby forming a pressed, dried collagen product; drying the pressed, lyophilized collagen product; thereby forming a dried, pressed collagen product; crosslinking the dried collagen product, thereby forming a crosslinked collagen product; drying the crosslinked collagen product, thereby forming a dried, crosslinked collagen product; pressing the dried, crosslinked collagen product, thereby forming a pressed, dried, crosslinked collagen product; and drying the pressed, dried, crosslinked collagen product, thereby forming a tri-layered collagen product. This method can produce a product that is structured such as the structure shown in FIG. 1.

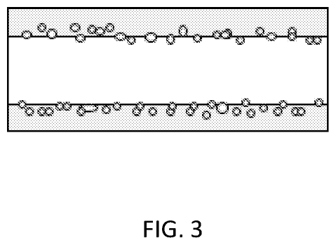

Disclosed are methods of making a tri-layer collagen composition comprising preparing a collagen slurry; drying at least a portion of the collagen slurry; optionally, pressing the dried collagen slurry to form a collagen base; perforating the first side of a collagen base, having a first side and a second side, thereby forming a perforated collagen base; adding bioactive glass to the first side of the perforated collagen base; coating the first side of the perforated collagen base comprising bioactive glass with the collagen slurry, thereby forming a single-coated collagen base; drying the single-coated collagen base; perforating the second side of the collagen base; adding bioactive glass to the second side of the perforated collagen base; coating the second side of the perforated collagen base comprising bioactive glass with the collagen slurry, thereby forming a double-coated collagen base; lyophilizing the double-coated collagen base, thereby forming a lyophilized collagen product; pressing the lyophilized collagen product, thereby forming a pressed, lyophilized collagen product; drying the pressed, lyophilized collagen product; forming a dried collagen product; crosslinking the dried collagen product, thereby forming a crosslinked collagen product: drying the crosslinked collagen product, thereby forming a dried, crosslinked collagen product; pressing the dried, crosslinked collagen product, thereby forming a pressed, dried, crosslinked collagen product; drying the pressed, dried, crosslinked collagen product, thereby forming a tri-layered collagen product. This method can produce a product that is structured such as the structure shown in FIG. 3.

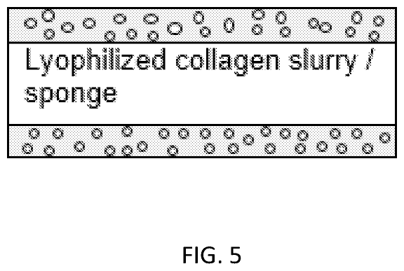

Disclosed are methods of making a tri-layer collagen composition comprising preparing a collagen slurry; lyophilizing at least a portion of the collagen slurry; pressing the lyophilized collagen slurry, thereby forming a collagen base; adding bioactive glass to a collagen slurry, thereby forming a bioactive glass collagen slurry mix; coating the first side of the collagen base, having a first side and a second side, with the bioactive glass collagen slurry mix, thereby forming a single-coated collagen base; drying the single-coated collagen base; coating the second side of the single-coated collagen base with the bioactive glass collagen slurry mix, thereby forming a double-coated collagen base; lyophilizing the double-coated collagen base, thereby forming a lyophilized collagen product; pressing the lyophilized collagen product, thereby forming a pressed, lyophilized collagen product; drying the pressed, lyophilized collagen product; thereby forming a dried collagen product; crosslinking the dried collagen product, thereby forming a crosslinked collagen product; drying the crosslinked collagen product, thereby forming a dried, crosslinked collagen product; pressing the dried, crosslinked collagen product, thereby forming a pressed, dried, crosslinked collagen product; and drying the pressed, dried, crosslinked collagen product, thereby forming a tri-layered collagen product. This method can produce a product that is structured such as the structure shown in FIG. 5.

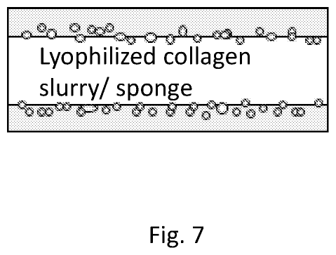

Disclosed are methods of making a tri-layer collagen composition comprising preparing a collagen slurry; lyophilizing at least a portion of the collagen slurry; pressing the lyophilized collagen slurry to form a collagen base; perforating the first side of a collagen base, having a first side and a second side, thereby forming a perforated collagen base; adding bioactive glass to the first side of the perforated collagen base; coating the first side of the perforated collagen base comprising bioactive glass with the collagen slurry, thereby forming a single-coated collagen base; drying the single-coated collagen base; perforating the second side of the collagen base; adding bioactive glass to the second side of the perforated collagen base: coating the second side of the perforated collagen base comprising bioactive glass with the collagen slurry, thereby forming a double-coated collagen base; lyophilizing the double-coated collagen base, thereby forming a lyophilized collagen product; pressing the lyophilized collagen product, thereby forming a pressed, lyophilized collagen product; drying the pressed, lyophilized collagen product, thereby forming a dried collagen product; crosslinking the dried collagen product; drying the crosslinked collagen product, thereby forming a dried, crosslinked collagen product; pressing the dried, crosslinked collagen product, thereby forming a pressed, dried, crosslinked collagen product; drying the pressed, dried, crosslinked collagen product, thereby forming a tri-layered collagen product. This method can produce a product that is structured such as the structure shown in FIG. 7.

In some aspects, several optional steps can also be performed, including, but not limited to, rinsing, cutting, packaging and sterilizing.

i. Preparing Collagen Slurry

The disclosed methods can comprise the step of preparing a collagen slurry. In some aspects, the collagen slurry comprises collagen and a buffer.

In some aspects, preparing the collagen slurry comprises obtaining a collagen sample. In some aspects, the collagen can be any of those disclosed herein, for example, marine, bovine, porcine, ovine, vegan, equine, mammalian, jellyfish, avian, reptilian, amphibian, bioengineered, or recombinant collagen.

In some aspects, the collagen slurry is a 0.9-1.25% (m/v) slurry. In some aspects, the collagen slurry can comprise a higher concentration which can create a denser end product. In some aspects, a higher concentration can be >1.10% m/v. In some aspects, a higher concentration can be up to about 2.5% m/v in the slurry. In some aspects, an increased density can lead to slower resorption of the entire composition. In some aspects, a higher concentration would result in a dry density on the order of 30-200 mg/cc in the sample. The collagen slurry has a lower percentage of collagen (m/v) compared to the final composition because eventually the collagen slurry can be lyophilized, or at least vacuum dried, which removes the majority of the water leaving a final composition resulting in a composition having 60-95%/wt collagen.

In some aspects, the method comprises adjusting the pH to about 7-10. In some aspects, the collagen slurry can further comprise a pH adjuster. Thus, in some aspects, the collagen used to prepare the slurry can be either acid buffer treated or alkaline buffer treated. In some aspects, preparing the collagen slurry comprises adding a pH adjuster, such as, but not limited to, HCl, citric acid, acetic acid, NaOH, or ammonia hydroxide. In some aspects, an alkaline collagen slurry can be more stable to the addition of bioactive glass.

In some aspects, instead of adding the pH adjuster to the slurry, the collagen itself can have its pH adjusted prior to making the slurry. For example, collagen can be immersed in an acid or alkaline bath and agitated for a given period of time in order to adjust the pH. In some aspects, the collagen is then rinsed with water to remove excess salts from the previous pH adjusting bath.

In some aspects, preparing the collagen slurry comprises homogenization. In some aspects, homogenizing can be performed while keeping the temperature below 20° C. results in more uniformity. In some aspects, an ice bath, cooling blanket, or temperature controlled bowl can be used to keep temperatures low. In some aspects, the homogenized slurry can be degassed, for example, via centrifugation or vacuum method. In some aspects, the homogenized slurry can be gently mixed following degassing since phase gravitational separation can occur

In some aspects, preparing the collagen slurry can include adding a crosslinking agent to the collagen and buffer. In some aspects, although a crosslinking agent is present in the collagen slurry, the step of crosslinking may not occur until after lyophilization. In some aspects, crosslinking can occur prior to the first lyophilization.

In some aspects, after preparing the collagen slurry, the collagen slurry can be poured into a container or mold. In some aspects, the container or mold can be used to prepare the collagen slurry in a specific shape and/or size. Thus, in some aspects, after the step of preparing a collagen slurry, the step of pouring the collagen slurry into a mold can be performed.

In some aspects, the composition can comprise a biocompatible buffer. For example, the composition can comprise citrate, acetate, phosphate, formate, tris, MES, and/or succinate buffer systems. The slurry can also comprise additional acids, bases, or salts for pH or handling adjustment, such as sodium hydroxide, potassium hydroxide, hydrochloric acid, acetic acid, sodium carbonate, sodium bicarbonate, sodium sulfate, sulfuric acid, sodium chloride, potassium chloride, sodium acetate, potassium sulfate, potassium carbonate, potassium bicarbonate, etc.

In some aspects, the buffer can be a pharmaceutically acceptable carrier. Thus, in some aspects, the disclosed compositions can comprise a pharmaceutically acceptable carrier. For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art.

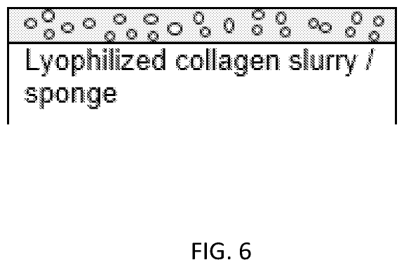

In some aspects, as shown in FIG. 5 and FIG. 6, the collagen slurry, after lyophilization, can be used as the collagen base. In some aspects, a separate collagen slurry can be prepared to be used as the collagen slurry used to coat the collagen base. Thus, more than one preparation of collagen slurry can be made during the disclosed methods. In some aspects, a prepared collagen slurry can be divided into several portions, wherein at least one portion is used to produce a collagen base made from lyophilized collagen slurry and/or at least one portion is used to coat (one or both sides of) the collagen base.

ii. Adding Bioactive Glass

In some aspects, the bioactive glass is 45S5, 58S, S53P4, or a Borate-formulation. In some aspects, the bioactive glass comprises 12-45% CaO, 0-65% SiO2, 0-30% Na2O, 2-17% P₂O₅, 0-7% MgO. 0-1% CaF₂, 0-60% B₂O₃, and 0-15% K₂O by mass percent.

In some aspects, the bioactive glass can be formed via melt or sol-gel glass processing methods. In some aspects, sol-gel derived glass can have dense or porous morphologies. In some aspects, porous morphology results in higher surface area, leading to faster rates of dissolution.

In some aspects, the bioactive glass has a particle size of about 10-1000 μm. For example, the composition can have a mean particle size of about 50. In some aspects, the bioactive glass is in the form of particles having a particle size of about 1-600 μm.

In some aspects, the disclosed composition comprise a non-porous bioactive glass. In some aspects, the disclosed composition comprise a porous bioactive glass. In some aspects, the bioactive glass has a porosity of up to 90%. In some aspects, the bioactive glass has pores ranging from about 1 to about 1000 microns. In some aspects, the bioactive glass has an average pore size of 100 microns plus or minus 50 microns, 200 microns plus or minus 50 microns. 300 microns plus or minus 50 microns, 400 microns plus or minus 50 microns. 500 microns plus or minus 50 microns, 600 microns plus or minus 50 microns, 700 microns plus or minus 50 microns.

In some aspects, the bioactive glass is in the form of irregular granules. In some aspects, the bioactive glass is in the form of approximately spherical particles. In some aspects, the bioactive glass is in the form of fibers.

In some aspects, the compositions comprise 1-30% bioactive glass. In some aspects, the compositions comprise 12-18% bioactive glass.

a. To the Collagen Slurry

In some aspects, the disclosed methods comprise the step of adding bioactive glass to the collagen slurry, forming a bioactive glass collagen slurry mix. In some aspects, the bioactive glass present in the collagen can range from 0.05%-15% by mass. In some aspects, the bioactive glass present in the collagen is about 1% by mass.

In some aspects, the bioactive glass is added to a collagen slurry that is not yet crosslinked. In some aspects, the bioactive glass is added to a collagen slurry that is already crosslinked.

In some aspects, a smaller size range of bioactive glass can be used for a homogenously dispersed monolayer composition. In some aspects, the bioactive glass is naturally buoyant and therefore no settling occurs via gravity.

In some aspects, a larger size range of bioactive glass can be used for a composition that is functionally graded through-the-thickness. In some aspects, larger bioactive glass can settle near bottom of the composition.

In some aspects, a combination of smaller and larger size ranges of bioactive glass are present in the disclosed compositions, with smaller particles dispersed throughout the composition and large particles that are pre-dominantly on one side due to settling.

In some aspects, the bioactive glass is distributed uniformly throughout the collagen slurry. In some aspects, the bioactive glass is distributed unevenly, or randomly, throughout the collagen slurry. In some aspects, at least one layer of the compositions comprise bioactive glass that is distributed uniformly throughout the collagen slurry. In some aspects, at least two layers of the compositions comprise bioactive glass that is distributed uniformly throughout the collagen slurry. In some aspects, the bioactive glass is distributed uniformly throughout the collagen in the first layer and the third layer.

b. To the Collagen Base

In some aspects, the bioactive glass can be added to the collagen base, not the collagen slurry. When adding to the collagen base, the collagen base can be prepped prior to the adding of the bioactive glass. In some aspects, the disclosed methods comprise the step of perforating the first side of a collagen base, having a first side and a second side, thereby forming a perforated collagen base. In some aspects, perforation can be performed by using a pinned roller tool. In some aspects, perforation can be performed using any tool that allows for perforating or puncturing the composition. In some aspects, the perforations do not extend all the way through the collagen base. In some aspects, only one side of the collagen base is perforated at a time. In some aspects, both sides of the collagen base can be perforated but only one side receives the bioactive glass at a time. In some aspects, the first step of perforating occurs prior to adding bioactive glass to the first side of the collagen base and the second step of perforating occurs after drying the single-coated collagen base. In some aspects, the perforating occurs after lyophilizing the collagen slurry to form a collagen base.

In some aspects, the collagen base is pure collagen. In some aspects, the collagen base is a lyophilized collagen slurry that may or may not be pressed which forms a sponge-like structure. Thus, in some aspects, the bioactive glass is added to a pressed, lyophilized collagen slurry that forms a collagen base and has been perforated.

In some aspects, the disclosed methods comprise the step of adding bioactive glass to the collagen base. In some aspects, adding bioactive glass to the collagen base can comprise sprinkling or lightly coating the perforated side of the collagen base with the bioactive glass.

In some aspects, the collagen base is dewatered prior to adding the bioactive glass. In some aspects, dewatered comprises the removal of excess moisture (e.g. water or a buffer).

iii. First Coating

In some aspects, the disclosed methods comprise the step of coating the first side of a collagen base, having a first side and a second side, with the bioactive glass collagen slurry mix, thus forming a single-coated collagen base. Or, if the bioactive glass was added to the collagen base, the disclosed methods comprise the step of coating the first side of the perforated collagen base comprising bioactive glass with the collagen slurry, thereby forming a single-coated collagen base.

In some aspects, a collagen base is a piece (e.g., sheet or layer) of collagen, usually at least 95% pure collagen. Thus, in some aspects, the collagen base does not comprise any bioactive glass. In some aspects, only the collagen base comprises bioactive glass. In some aspects, the collagen base is purified collagen. In some aspects, the purified collagen is purified type I collagen derived from bovine flexor tendon. In some aspects, a collagen base is collagen purchased from a vendor, such as Type 1 Bovine Flexor Tendon Collagen sheets.

In some aspects, only the first side of the collagen base can be coated prior to the first drying step.

In some aspects, the collagen base is dewatered prior to coating. In some aspects, dewatered comprises the removal of excess moisture (e.g. water or a buffer).

iv. First Drying

In some aspects, the disclosed methods comprise a first drying step comprising drying the single-coated collagen base. Because the first drying step involves the single-coated collagen base, the drying always occurs after coating the first side of a collagen base with the collagen slurry mix (whether the collagen base has the bioactive glass or the collagen slurry has the bioactive glass).

In some aspects, the drying occurs by placing the collagen base on a surface with the coated side up.

In some aspects, the drying step can include lyophilization. In some aspects, the lyophilization can comprise freezing the single-coated collagen base and then using a vacuum to dry out the water. In some aspects, the vacuum drying can be performed at the same temperature as the freezing or can be performed at a temperature 1°, 5°, 10°, 15°, 20°, 25°, 30°, 35°, 40°, 45°, or 50° C. higher than the temperature of the freezing step. In some aspects, the vacuum drying step after freezing can be carried out for less than 10 hours. In some aspects, the vacuum drying step can be carried out for 10, 12, 14, 16, 18, 20, or 24 hours. In some aspects, the vacuum drying step can be carried out for 24, 48 or 72 hours. In some aspects, the vacuum drying step can be carried out for 12-144 hours.

In some aspects, the drying can comprise lyophilization, vacuum drying, air drying, and/or critical point drying.

v. Second Coating

In some aspects, the disclosed methods comprise the step of coating the second side of the collagen base with a bioactive glass collagen slurry mix, thus forming a double-coated collagen base. In some aspects, coating the second side of the collagen base can be referred to as coating the single-coated collagen base on the collagen base side, not the bioactive glass collagen slurry mix side.

Or, if the bioactive glass was added to the collagen base, the disclosed methods comprise the step of coating the second side of the perforated collagen base comprising bioactive glass with the collagen slurry, thereby forming a double-coated collagen base. In the case of coating a second side of a perforated collagen base with bioactive glass, the second side of the collagen base can be perforated after drying the single-coated collagen base. In some aspects, both the first side and second side of the collagen base can be perforated prior to coating the first side.

In some aspects, when coating the second side of the collagen base, the single-coated collagen base is placed on a surface with the coated side down so that the second side of the collagen base (e.g., the side not yet coated) is available for coating.

In some aspects, the collagen slurry mix (with or without bioactive glass) coated on the second side is the same as the collagen slurry mix (with or without bioactive glass) coated on the first side of the collagen base. In some aspects, the collagen slurry mix (with or without bioactive glass) coated on the second side is the different from the collagen slurry mix (with or without bioactive glass) coated on the first side of the collagen base. Thus, in some aspects, a new collagen slurry mix (with or without bioactive glass) is prepared prior to the coating of the second side.

In some aspects, the same collagen slurry mix used for the first coating step can be used for the second coating step.

vi. Second Drying

In some aspects, the disclosed methods comprise a second drying step comprising drying the double-coated collagen base, thereby forming a dried collagen product. Because the second drying step involves the double-coated collagen base, the drying always occurs after coating of both the first side and the second side of a collagen base with the bioactive glass collagen slurry mix.

In some aspects, the drying occurs by placing the double-coated collagen base on a surface with the freshly coated second side up. Thus, in some aspects, the previously dried collagen has the bioactive glass collagen slurry mix side down because it is already dried and does not stick to the surface.

In some aspects, the second drying step can be a second lyophilizing step since the first drying step can be lyophilization. In some aspects, the drying step can include lyophilization. In some aspects, the lyophilization can comprise freezing the double-coated collagen base and then using a vacuum to dry out the water. Thus, in some aspects, the dried collagen product can be a lyophilized collagen product. In some aspects, the vacuum drying can be performed at the same temperature as the freezing or can be performed at a temperature 1°. 5°, 10°, 15°, 20°, 25°, 30°. 35°, 40°, 45°, or 50° C. higher than the temperature of the freezing step. In some aspects, the vacuum drying step after freezing can be carried out for less than 10 hours. In some aspects, the vacuum drying step can be carried out for 10, 12, 14, 16, 18, 20, or 24 hours. In some aspects, the vacuum drying step can be carried out for 24, 48 or 72 hours. In some aspects, the vacuum drying step can be carried out for 12-144 hours.

In some aspects, the drying can comprise lyophilization, vacuum drying, air drying, and/or critical point drying.

In some aspects, it is possible to coat the first side and the second side at the same time and thus, dry them at the same time. Thus, in some aspects, the second coating step is part of the first coating step and the second drying step is part of the first drying step (i.e., one coating and one drying after coating step).

vii. First Pressing

In some aspects, the disclosed methods comprise a step of pressing the dried collagen product, thereby forming a pressed, dried collagen product. Because the drying step is often lyophilization, the step of pressing can be pressing the lyophilized collagen product, thereby forming a pressed, lyophilized collagen product.

In some aspects, this is not the first pressing step. If the collagen base is produced by lyophilizing a collagen slurry, it is then pressed to form the collagen base. Thus, this step of pressing would be the second pressing step.

In some aspects, pressing the dried double-coated collagen base locks in a shape of the dried double-coated collagen base. For example, the shape can be, but is not limited to, square, rectangle, oval, or circle.

In some aspects, pressing the dried double-coated collagen base results in the dried double-coated collagen base having a thickness of 1 mm. In some aspects, the result of pressing is a thickness of 0.1 to 1 mm. In some aspects, the thickness of the dried double-coated collagen base before pressing can be between 2 to 5 mm. 2 to 10 mm, or 3 to 8 mm.

In some aspects, the dried coated collagen base is humidified to 15%-35% w/w water. Water can act as a plasticizer, aiding the structure to lock its shape after pressing.

In some aspects, the humidified structure is pressed by running through a manual roller press machine. In this machine, the collagen sheet is placed between two flat polymer screens that are pressed by two rollers. In some aspects, it can be run through twice to make sure the collagen base is thin all around.

viii. Third Drying

In some aspects, the disclosed methods comprise a step of drying the pressed, dried collagen product: thereby forming a dried, pressed collagen product. In some aspects, this step is the third drying step.

In some aspects, the third drying step is not lyophilization. In some aspects, the third drying step comprises vacuum drying. In some aspects, the third drying step can be carried out for less than 10 hours. In some aspects, the third drying step can be carried out for 10, 12, 14, 16, 18, 20, or 24 hours. In some aspects, the third drying step can be carried out for 24, 48 or 72 hours. In some aspects, the third drying step can be carried out for 12-144 hours.

In some aspects, the drying can comprise lyophilization, vacuum drying, air drying, and/or critical point drying.

ix. Crosslinking

In some aspects, the disclosed methods comprise a step of crosslinking the dried, pressed collagen product, thereby forming a crosslinked collagen product. In some aspects, this step crosslinks the collagen base and the layers comprising the bioactive glass.

In some aspect, the crosslinking step comprises adding a crosslinking agent to the dried, pressed collagen product. In some aspects, the crosslinking agent has been added to the product at any one of the above steps. In some aspects, the crosslinking step can occur at any step or at multiple steps. At some aspects, the crosslinking agent can be added at any step but the crosslinking is not activated until after the third drying step.

In some aspects, a crosslinking agent is mixed in, or added to, the collagen slurry during preparation of the collagen slurry. In some aspects, a cross-linking agent can be added after one or more of the drying steps. In some aspects, heat treatment under a vacuum can be used to crosslink the collagen.

In some aspects, the crosslinking can be chemical. Thus, in some aspects, the crosslinking agent used during the crosslinking step can be, but is not limited to, glutaraldehyde, citric acid, glycine, lysine, glutamic acid, serine, trilysine, transglutaminase, ethylene oxide, phosphate (cross linking aid), tris, carbodiimide, or EDC-NHS. In some aspects, the crosslinking agent is glutaraldehyde, wherein the glutaraldehyde is in a final concentration of about 0.01% to 0.1% w/w. In some aspects, chemical crosslinking comprises contacting the dried collagen product with a chemical, such as, glutaraldehyde. In some aspects, crosslinking comprises soaking the dried collagen product in a solution comprising 0.01% w/w to 0.1% w/w of a chemical, such as, glutaraldehyde.

In some aspects, no crosslinking agent is needed for crosslinking. In some aspects, the crosslinking step can occur using methods, such as, but not limited to, UV, ethylene oxide, glutaraldehyde vapor, enzymatic crosslinking, dehydrothermal treatment, physicochemical crosslinking, and physicothermal crosslinking Thus, any known method of cross-linking can be used in the disclosed methods.

In some aspects, an amount of cross-linking agent is added to the collagen sample to result in 0.01% to 5% (dry weight) of the final product.

x. Fourth Drying

In some aspects, the disclosed methods comprise a step of drying the crosslinked collagen product; thereby forming a dried, crosslinked collagen product. In some aspects, this step is the fourth drying step.

In some aspects, the fourth drying step can be a lyophilizing step or a vacuum drying step. In some aspects, the fourth drying step can comprise both lyophilizing and vacuum drying. In some aspects, the vacuum drying step can be carried out for 10, 12, 14, 16, 18, 20, or 24 hours. In some aspects, the vacuum drying step can be carried out for 24, 48 or 72 hours. In some aspects, the vacuum drying step can be carried out for 12-144 hours.

In some aspects, the drying can comprise lyophilization, vacuum drying, air drying, and/or critical point drying.

xi. Second Pressing

In some aspects, the disclosed methods comprise a step of pressing the dried, crosslinked collagen product, thereby forming a pressed, dried, crosslinked collagen product.

In some aspects, pressing the dried, crosslinked collagen product retains the shape obtained after the first pressing step.

If, as stated above, the first pressing step is really the second pressing step then, in some aspects, this step is the third pressing step.

In some aspects, pressing the dried, crosslinked collagen product results in the dried, crosslinked collagen product having a thickness of 1 mm. In some aspects, the result of pressing is a thickness of 0.1 to 1 mm.

In some aspects, the second pressing step can be performed identical to the first pressing step.

xii. Fifth Drying

In some aspects, the disclosed methods comprise a step of drying the pressed, dried, crosslinked collagen product, thereby forming a tri-layered collagen product.

In some aspects, the fifth drying step can be a lyophilizing step or a vacuum drying step. In some aspects, the fifth drying step can comprise both lyophilizing and vacuum drying. In some aspects, the vacuum drying step can be carried out for 10, 12, 14, 16, 18, 20, or 24 hours. In some aspects, the vacuum drying step can be carried out for 24, 48 or 72 hours. In some aspects, the vacuum drying step can be carried out for 12-144 hours.

In some aspects, the drying can comprise lyophilization, vacuum drying, air drying, and/or critical point drying.

xiii. Additional Steps

In some aspects, the disclosed methods further comprising a rinsing step after the crosslinking step and before the fourth drying step. In some aspects, the rinsing step reduces the residual chemicals, such as, aldehydes (e.g., from the crosslinking). In some aspects, rinsing comprises rinsing the dried collagen product with sodium borohydride.

In some aspects, the rinsing step comprises a water rinsing step. For example, the product can be rinsed with water before and/or after rinsing with sodium borohydride.

In some aspects, the rinsing step is followed by a drying step, for example, the fourth drying step.

In some aspects, the disclosed methods further comprise cutting the final product. In some aspects, cutting the final product allows for specific sizes or shapes to be produced. In some aspects, the cutting can occur after the last drying step. In some aspects, cutting can occur at any step. For example, the collagen can be cut at any step of the disclosed methods.

In some aspects, the disclosed methods further comprise packaging the final product. In some aspects, the packaging can be, but is not limited to, Tyvek-PET heat sealed packaging, heat sealed foil pouches, peel packs, or trays with foil or tyvek lids. In some aspects, any sterile barrier system would be suitable for packaging.

In some aspects, the disclosed methods further comprise sterilizing the final product. In some aspects, sterilization can occur just before packaging. In some aspects, sterilization can occur after packaging.

### 2. Bilayer

Disclosed are methods of making a bilayer collagen composition comprising preparing a collagen slurry; adding bioactive glass, coating a first side of a collagen base with the bioactive glass collagen slurry mix, lyophilizing and crosslinking. Examples of each of these steps is described in more detail below and the order of each of these steps can be changed.

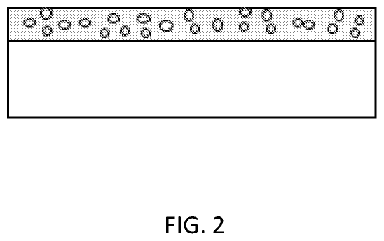

Disclosed are methods of making a bilayer collagen composition comprising preparing a collagen slurry; adding bioactive glass to the collagen slurry, forming a bioactive glass collagen slurry mix: coating one side of a collagen base with the bioactive glass collagen slurry mix, forming a single-coated collagen base; drying the single-coated collagen base, thereby forming a dried collagen product; pressing the dried collagen product, thereby forming a pressed, dried collagen product: drying the pressed, dried collagen product, thereby forming a dried collagen product: crosslinking the dried collagen product, thereby forming a crosslinked collagen product: drying the crosslinked collagen product, thereby forming a dried, crosslinked collagen product; pressing the dried, crosslinked collagen product, thereby forming a pressed, dried, crosslinked collagen product; drying the pressed, dried, crosslinked collagen product, thereby forming a bilayered collagen product. This method can produce a product that is structured as shown in FIG. 2.

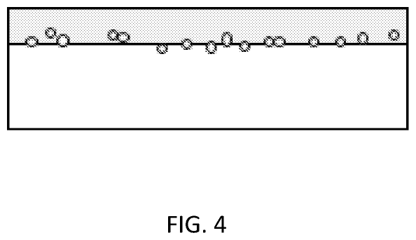

Disclosed are methods of making a bilayer collagen composition comprising preparing a collagen slurry; drying at least a portion of the collagen slurry; optionally, pressing the dried collagen slurry to form a collagen base; perforating one side of a collagen base; adding bioactive glass to the perforated side of the collagen base; coating the perforated collagen base side comprising bioactive glass with the collagen slurry, thereby forming a single-coated collagen base; drying the single-coated collagen base: thereby forming a dried, collagen product; pressing the dried collagen product, thereby forming a pressed, dried collagen product; drying the pressed, dried collagen product, thereby forming a dried collagen product; crosslinking the dried collagen product, thereby forming a crosslinked collagen product: drying the crosslinked collagen product, thereby forming a dried, crosslinked collagen product; pressing the dried, crosslinked collagen product, thereby forming a pressed, dried, crosslinked collagen product; drying the pressed, dried, crosslinked collagen product, thereby forming a tri-layered collagen product. This method can produce a product that is structured as shown in FIG. 4.

Disclosed are methods of making a bilayer collagen composition comprising preparing a collagen slurry; lyophilize at least a portion of the collagen slurry: pressing the lyophilized collagen slurry to form a collagen base; adding bioactive glass to a collagen slurry, forming a bioactive glass collagen slurry mix; coating one side of the collagen base with the bioactive glass collagen slurry mix, thereby forming a single-coated collagen base; drying the single-coated collagen base: lyophilizing the single-coated collagen base, thereby forming a lyophilized collagen product; pressing the lyophilized collagen product, thereby forming a pressed, lyophilized collagen product; drying the pressed, lyophilized collagen product, thereby forming a dried collagen product; crosslinking the dried collagen product, thereby forming a crosslinked collagen product: drying the crosslinked collagen product, thereby forming a dried, crosslinked collagen product; pressing the dried, crosslinked collagen product, thereby forming a pressed, dried, crosslinked collagen product; and drying the pressed, dried, crosslinked collagen product, thereby forming a bilayered collagen product. This method can produce a product that is structured as shown in FIG. 6.

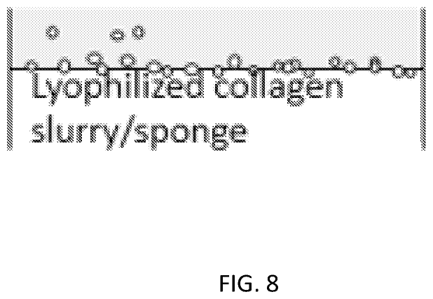

Disclosed are methods of making a bilayer collagen composition comprising preparing a collagen slurry; lyophilizing at least a portion of the collagen slurry; pressing the lyophilized collagen slurry to form a collagen base; perforating one side of a collagen base; adding bioactive glass to the perforated collagen base; coating the side of the perforated collagen base comprising bioactive glass with the collagen slurry, forming a single-coated collagen base; drying the single-coated collagen base; lyophilizing the single-coated collagen base, thereby forming a lyophilized collagen product; pressing the lyophilized collagen product, thereby forming a pressed, lyophilized collagen product: drying the pressed, lyophilized collagen product, thereby forming a dried collagen product; crosslinking the dried collagen product, thereby forming a crosslinked collagen product; drying the crosslinked collagen product, thereby forming a dried, crosslinked collagen product; pressing the dried, crosslinked collagen product, thereby forming a pressed, dried, crosslinked collagen product; drying the pressed, dried, crosslinked collagen product, thereby forming a bilayered collagen product. This method can produce a product that is structured as shown in FIG. 8.

In some aspects, a bilayer collagen product can be produced following the same method as forming the trilayer collagen product with the exception of not performing the second coating step of the trilayer method.

i. Preparing Collagen Slurry

The disclosed methods can comprise the step of preparing a collagen slurry. In some aspects, the collagen slurry comprises collagen and a buffer.

In some aspects, preparing the collagen slurry comprises obtaining a collagen sample. In some aspects, the collagen can be any of those disclosed herein, for example, marine, bovine, porcine, ovine, vegan, equine, mammalian, jellyfish, avian, reptilian, amphibian, bioengineered, or recombinant collagen.

In some aspects, the collagen slurry is a 0.9-1.25% (m/v) slurry. In some aspects, the collagen slurry can comprise a higher concentration which can create a denser end product. In some aspects, a higher concentration can be >1.10% m/v. In some aspects, a higher concentration can be up to about 2.5% m/v in the slurry. In some aspects, an increased density can lead to slower resorption of the entire composition. In some aspects, a higher concentration would result in a dry density on the order of 30-200 mg/cc in the sample. The collagen slurry has a lower percentage of collagen (m/v) compared to the final composition because eventually the collagen slurry can be lyophilized, or at least vacuum dried, which removes the majority of the water leaving a final composition resulting in a composition having 60-95%/wt collagen.

In some aspects, the method comprises adjusting the pH to about 7-10. In some aspects, the collagen slurry can further comprise a pH adjuster. Thus, in some aspects, the collagen used to prepare the slurry can be either acid buffer treated or alkaline buffer treated. In some aspects, preparing the collagen slurry comprises adding a pH adjuster, such as, but not limited to, HCl, citric acid, acetic acid, NaOH, or ammonia hydroxide. In some aspects, an alkaline collagen slurry can be more stable to the addition of bioactive glass.

In some aspects, instead of adding the pH adjuster to the slurry, the collagen itself can have its pH adjusted prior to making the slurry. For example, collagen can be immersed in an acid or alkaline bath and agitated for a given period of time in order to adjust the pH. In some aspects, the collagen is then rinsed with water to remove excess salts from the previous pH adjusting bath.

In some aspects, preparing the collagen slurry comprises homogenization. In some aspects, homogenizing can be performed while keeping the temperature below 20° C. results in more uniformity. In some aspects, an ice bath, cooling blanket, or temperature controlled bowl can be used to keep temperatures low. In some aspects, the homogenized slurry can be degassed, for example, via centrifugation or vacuum method. In some aspects, the homogenized slurry can be gently mixed following degassing since phase gravitational separation can occur

In some aspects, preparing the collagen slurry can include adding a crosslinking agent to the collagen and buffer. In some aspects, although a crosslinking agent is present in the collagen slurry, the step of crosslinking may not occur until after lyophilization. In some aspects, crosslinking can occur prior to the first lyophilization.

In some aspects, after preparing the collagen slurry, the collagen slurry can be poured into a container or mold. In some aspects, the container or mold can be used to prepare the collagen slurry in a specific shape and/or size. Thus, in some aspects, after the step of preparing a collagen slurry, the step of pouring the collagen slurry into a mold can be performed.

In some aspects, the composition can comprise a biocompatible buffer. For example, the composition can comprise citrate, acetate, phosphate, formate, tris, MES, and/or succinate buffer systems. The slurry can also comprise additional acids, bases, or salts for pH or handling adjustment, such as sodium hydroxide, potassium hydroxide, hydrochloric acid, acetic acid, sodium carbonate, sodium bicarbonate, sodium sulfate, sulfuric acid, sodium chloride, potassium chloride, sodium acetate, potassium sulfate, potassium carbonate, potassium bicarbonate, etc.

In some aspects, the buffer can be a pharmaceutically acceptable carrier. Thus, in some aspects, the disclosed compositions can comprise a pharmaceutically acceptable carrier. For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art.

In some aspects, as shown in FIG. 5 and FIG. 6, the collagen slurry, after lyophilization, can be used as the collagen base. In some aspects, a separate collagen slurry can be prepared to be used as the collagen slurry used to coat the collagen base. Thus, more than one preparation of collagen slurry can be made during the disclosed methods. In some aspects, a prepared collagen slurry can be divided into several portions, wherein at least one portion is used to produce a collagen base made from lyophilized collagen slurry and/or at least one portion is used to coat one side of the collagen base.

ii. Adding Bioactive Glass

In some aspects, the bioactive glass is 45S5, 58S, S53P4, or a Borate-formulation. In some aspects, the bioactive glass comprises 12-45% CaO, 0-65% SiO2, 0-30% Na2O, 2-17% P₂O₅, 0-7% MgO, 0-1% CaF₂, 0-60% B₂O₃, and 0-15% K₂O by mass percent.

In some aspects, the bioactive glass can be formed via melt or sol-gel glass processing methods. In some aspects, sol-gel derived glass can have dense or porous morphologies. In some aspects, porous morphology results in higher surface area, leading to faster rates of dissolution.

In some aspects, the bioactive glass has a particle size of about 10-1000 μm. For example, the composition can have a mean particle size of about 50. In some aspects, the bioactive glass is in the form of particles having a particle size of about 1-600 μm.

In some aspects, the disclosed composition comprise a non-porous bioactive glass. In some aspects, the disclosed composition comprise a porous bioactive glass. In some aspects, the bioactive glass has a porosity of up to 90%. In some aspects, the bioactive glass has pores ranging from about 1 to about 1000 microns. In some aspects, the bioactive glass has an average pore size of 100 microns plus or minus 50 microns, 200 microns plus or minus 50 microns. 300 microns plus or minus 50 microns, 400 microns plus or minus 50 microns. 500 microns plus or minus 50 microns, 600 microns plus or minus 50 microns, 700 microns plus or minus 50 microns.

In some aspects, the bioactive glass is in the form of irregular granules. In some aspects, the bioactive glass is in the form of approximately spherical particles. In some aspects, the bioactive glass is in the form of fibers.

In some aspects, the compositions comprise 1-30% bioactive glass. In some aspects, the compositions comprise 12-18% bioactive glass.

a. To the collagen slurry

In some aspects, the disclosed methods comprise the step of adding bioactive glass to the collagen slurry, forming a bioactive glass collagen slurry mix. In some aspects, the bioactive glass present in the collagen can range from 0.05%-15% by mass. In some aspects, the bioactive glass present in the collagen is about 1% by mass.

In some aspects, the bioactive glass is added to a collagen slurry that is not yet crosslinked. In some aspects, the bioactive glass is added to a collagen slurry that is already crosslinked.

In some aspects, a smaller size range of bioactive glass can be used for a homogenously dispersed monolayer composition. In some aspects, the bioactive glass is naturally buoyant and therefore no settling occurs via gravity.

In some aspects, a larger size range of bioactive glass can be used for a composition that is functionally graded through-the-thickness. In some aspects, larger bioactive glass can settle near bottom of the composition.

In some aspects, a combination of smaller and larger size ranges of bioactive glass are present in the disclosed compositions, with smaller particles dispersed throughout the composition and large particles that are pre-dominantly on one side due to settling.

In some aspects, the bioactive glass is distributed uniformly throughout the collagen slurry. In some aspects, the bioactive glass is distributed unevenly, or randomly, throughout the collagen slurry. In some aspects, at least one layer of the compositions comprise bioactive glass that is distributed uniformly throughout the collagen slurry. In some aspects, at least two layers of the compositions comprise bioactive glass that is distributed uniformly throughout the collagen slurry.

b. To the Collagen Base

In some aspects, the bioactive glass can be added to the collagen base, not the collagen slurry. When adding to the collagen base, the collagen base can be prepped prior to the adding of the bioactive glass. In some aspects, the disclosed methods comprise the step of perforating the first side of a collagen base, having a first side and a second side, thereby forming a perforated collagen base. In some aspects, perforation can be performed by using a pinned roller tool. In some aspects, perforation can be performed using any tool that allows for perforating or puncturing the composition. In some aspects, the perforations do not extend all the way through the collagen base. In some aspects, only one side of the collagen base is perforated. In some aspects, perforating occurs prior to adding bioactive glass to the first side of the collagen base. In some aspects, the perforating occurs after lyophilizing the collagen slurry to form a collagen base.

In some aspects, the collagen base is pure collagen. In some aspects, the collagen base is a lyophilized collagen slurry that may or may not be pressed which forms a sponge-like structure. Thus, in some aspects, the bioactive glass is added to a pressed, lyophilized collagen slurry that forms a collagen base that has been perforated.

In some aspects, the disclosed methods comprise the step of adding bioactive glass to the collagen base. In some aspects, adding bioactive glass to the collagen base can comprise sprinkling or lightly coating the perforated side of the collagen base with the bioactive glass.

In some aspects, the collagen base is dewatered prior to adding the bioactive glass. In some aspects, dewatered comprises the removal of excess moisture (e.g. water or a buffer).

iii. Coating

In some aspects, the disclosed methods comprise the step of coating one side of a collagen base with the bioactive glass collagen slurry mix, thereby forming a single-coated collagen base. Or, if the bioactive glass was added to the collagen base, the disclosed methods comprise the step of coating the perforated collagen base side comprising bioactive glass with the collagen slurry, thereby forming a single-coated collagen base.

In some aspects, a collagen base is a piece (e.g., sheet or layer) of collagen, usually at least 95% pure collagen. Thus, in some aspects, the collagen base does not comprise any bioactive glass. In some aspects, the collagen base is purified collagen. In some aspects, the purified collagen is purified type I collagen derived from bovine flexor tendon. In some aspects, a collagen base is collagen purchased from a vendor, such as Type 1 Bovine Flexor Tendon Collagen sheets.

In some aspects, the collagen base is dewatered prior to coating. In some aspects, dewatered comprises the removal of excess moisture (e.g. water or a buffer).

iv. First Drying

In some aspects, the disclosed methods comprise a first drying step comprising drying the single-coated collagen base. Because the first drying step involves the single-coated collagen base, the drying always occurs after coating one side of a collagen base with the bioactive glass collagen slurry mix.

In some aspects, the drying occurs by placing the collagen base on a surface with the bioactive glass collagen slurry mix side up.

In some aspects, the drying step can include lyophilization. In some aspects, the lyophilization can comprise freezing the single-coated collagen base and then using a vacuum to dry out the water. In some aspects, the vacuum drying can be performed at the same temperature as the freezing or can be performed at a temperature 1°, 5°, 10°, 15°, 20°, 25°, 30°, 35°, 40°, 45°, or 50° C. higher than the temperature of the freezing step. In some aspects, the vacuum drying step after freezing can be carried out for less than 10 hours. In some aspects, the vacuum drying step can be carried out for 10, 12, 14, 16, 18, 20, or 24 hours. In some aspects, the vacuum drying step can be carried out for 24, 48 or 72 hours. In some aspects, the vacuum drying step can be carried out for 12-144 hours.

In some aspects, the drying can comprise lyophilization, vacuum drying, air drying, and/or critical drying.

v. First Pressing

In some aspects, the disclosed methods comprise a step of pressing the dried collagen product, thereby forming a pressed, dried collagen product. Because the drying step is often lyophilization, the step of pressing can be pressing the lyophilized collagen product, thereby forming a pressed, lyophilized collagen product.

In some aspects, this is not the first pressing step. If the collagen base is produced by lyophilizing a collagen slurry, it is then pressed to form the collagen base. Thus, this step of pressing would be the second pressing step.

In some aspects, pressing the dried double-coated collagen base locks in a shape of the dried double-coated collagen base. For example, the shape can be, but is not limited to, square, rectangle, oval, or circle.

In some aspects, pressing the dried single-coated collagen base results in the dried single-coated collagen base having a thickness of <1 mm. In some aspects, the result of pressing is a thickness of 0.1 to 1 mm. In some aspects, the thickness of the dried single-coated collagen base before pressing can be between 2 to 5 mm, 2 to 10 mm, or 3 to 8 mm.

In some aspects, the dried coated collagen base is humidified to 15%-35% w/w water. Water can act as a plasticizer, aiding the structure to lock its shape after pressing.

In some aspects, the humidified structure is pressed by running through a manual roller press machine. In this machine, the collagen sheet is placed between two flat polymer screens that are pressed by two rollers. In some aspects, it can be run through twice to make sure the collagen base is thin all around.

vi. Second Drying

In some aspects, the disclosed methods comprise a step of drying the pressed, dried collagen product; thereby forming a dried, pressed collagen product. In some aspects, this step is the second drying step and occurs after a first drying step and a pressing step.

In some aspects, the second drying step is not lyophilization. In some aspects, the second drying step comprises vacuum drying. In some aspects, the second drying step can be carried out for less than 10 hours. In some aspects, the second drying step can be carried out for 10, 12, 14, 16, 18, 20, or 24 hours. In some aspects, the second drying step can be carried out for 24, 48 or 72 hours. In some aspects, the second drying step can be carried out for 12-144 hours.

In some aspects, the drying can comprise lyophilization, vacuum drying, air drying, and/or critical point drying.

vii. Crosslinking

In some aspects, the disclosed methods comprise a step of crosslinking the dried, pressed collagen product, thereby forming a crosslinked collagen product. In some aspects, this step crosslinks the collagen base and the layer comprising the bioactive glass.

In some aspect, the crosslinking step comprises adding a crosslinking agent to the dried, pressed collagen product. In some aspects, the crosslinking agent has been added to the product at any one of the above steps. In some aspects, the crosslinking step can occur at any step or at multiple steps. At some aspects, the crosslinking agent can be added at any step but the crosslinking is not activated until after the second drying step.

In some aspects, a crosslinking agent is mixed in, or added to, the collagen slurry during preparation of the collagen slurry. In some aspects, a cross-linking agent can be added after one or more of the drying steps. In some aspects, heat treatment under a vacuum can be used to crosslink the collagen.

In some aspects, the crosslinking can be chemical. Thus, in some aspects, the crosslinking agent used during the crosslinking step can be, but is not limited to, glutaraldehyde, citric acid, glycine, lysine, glutamic acid, serine, trilysine, transglutaminase, ethylene oxide, phosphate (cross linking aid), tris, carbodiimide, or EDC-NHS. In some aspects, the crosslinking agent is glutaraldehyde, wherein the glutaraldehyde is in a final concentration of about 0.01% to 0.1% w/w. In some aspects, chemical crosslinking comprises contacting the dried collagen product with a chemical, such as, glutaraldehyde. In some aspects, crosslinking comprises soaking the dried collagen product in a solution comprising 0.01% w/w to 0.1% w/w of a chemical, such as, glutaraldehyde.

In some aspects, no crosslinking agent is needed for crosslinking. In some aspects, the crosslinking step can occur using methods, such as, but not limited to, UV, ethylene oxide, glutaraldehyde vapor, enzymatic crosslinking, dehydrothermal treatment, physicochemical crosslinking, and physicothermal crosslinking Thus, any known method of cross-linking can be used in the disclosed methods.

In some aspects, an amount of cross-linking agent is added to the collagen sample to result in 0.01% to 5% (dry weight) of the final product.

viii. Third Drying

In some aspects, the disclosed methods comprise a step of drying the crosslinked collagen product; thereby forming a dried, crosslinked collagen product. In some aspects, this step is the third drying step.

In some aspects, the third drying step can be a lyophilizing step or a vacuum drying step. In some aspects, the third drying step can comprise both lyophilizing and vacuum drying. In some aspects, the vacuum drying step can be carried out for 10, 12, 14, 16, 18, 20, or 24 hours. In some aspects, the vacuum drying step can be carried out for 24, 48 or 72 hours. In some aspects, the vacuum drying step can be carried out for 12-144 hours.

In some aspects, the drying can comprise lyophilization, vacuum drying, air drying, and/or critical point drying.

ix. Second Pressing

In some aspects, the disclosed methods comprise a step of pressing the dried, crosslinked collagen product, thereby forming a pressed, dried, crosslinked collagen product.

In some aspects, pressing the dried, crosslinked collagen product retains the shape obtained after the first pressing step.

If, as stated above, the first pressing step is really the second pressing step then, in some aspects, this step is the third pressing step.

In some aspects, pressing the dried, crosslinked collagen product results in the dried, crosslinked collagen product having a thickness of 1 mm. In some aspects, the result of pressing is a thickness of 0.1 to 1 mm.

In some aspects, the second pressing step can be performed identical to the first pressing step.

x. Fourth Drying

In some aspects, the disclosed methods comprise a step of drying the pressed, dried, crosslinked collagen product, thereby forming a bilayered collagen product.

In some aspects, the fourth drying step can be a lyophilizing step or a vacuum drying step. In some aspects, the fourth drying step can comprise both lyophilizing and vacuum drying. In some aspects, the vacuum drying step can be carried out for 10, 12, 14, 16, 18, 20, or 24 hours. In some aspects, the vacuum drying step can be carried out for 24, 48 or 72 hours. In some aspects, the vacuum drying step can be carried out for 12-144 hours.

In some aspects, the drying can comprise lyophilization, vacuum drying, air drying, and/or critical point drying.

xi. Additional Steps

In some aspects, the disclosed methods further comprising a rinsing step after the crosslinking step and before the third drying step. In some aspects, the rinsing step reduces the residual chemicals, such as, aldehydes (e.g., from the crosslinking). In some aspects, rinsing comprises rinsing the dried collagen product with sodium borohydride.

In some aspects, the rinsing step comprises a water rinsing step. For example, the product can be rinsed with water before and/or after rinsing with sodium borohydride.

In some aspects, the rinsing step is followed by a drying step, for example, the third drying step.

In some aspects, the disclosed methods further comprise cutting the final product. In some aspects, cutting the final product allows for specific sizes or shapes to be produced. In some aspects, the cutting can occur after the last drying step. In some aspects, cutting can occur at any step. For example, the collagen can be cut at any step of the disclosed methods.

In some aspects, the disclosed methods further comprise packaging the final product. In some aspects, the packaging can be, but is not limited to, Tyvek-PET heat sealed packaging, heat sealed foil pouches, peel packs, or trays with foil or tyvek lids. In some aspects, any sterile barrier system would be suitable for packaging.

In some aspects, the disclosed methods further comprise sterilizing the final product. In some aspects, sterilization can occur just before packaging. In some aspects, sterilization can occur after packaging.

### 3. Monolayer

Disclosed are methods of making a monolayer collagen composition comprising preparing a collagen slurry and adding bioactive glass. In some aspects, disclosed are methods of making a composition comprising preparing a collagen slurry; lyophilizing the collagen slurry to create a lyophilized collagen sample; cross-linking the collagen slurry before and/or after lyophilization; adding bioactive glass to a cross-linked lyophilized collagen sample.

In some aspects, disclosed are methods of making a composition comprising preparing a collagen slurry and adding bioactive glass to a cross-linked lyophilized collagen sample. In some aspects, the method can comprise lyophilizing the collagen slurry to create a lyophilized collagen sample and/or cross-linking the collagen slurry before and/or after lyophilization. In some aspects, the lyophilization can occur before or after adding the bioactive glass.

In some aspects, the collagen slurry can be prepared as described throughout. The bioactive glass can be added as described throughout. For example, the bioactive glass can be uniformly or randomly distributed.

In some aspects, the collagen slurry:bioactive glass mixture can be dried. Any of the drying methods described throughout can be used.

### F. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising collagen and bioactive glass. In some aspects, the kits can further comprise a crosslinking agent and/or buffers.

### EXAMPLES

### A. Example 1: Collagen/Bioglass Dental Membrane

Disclosed is a composition that is a soft, white to off-white, pliable, non-friable tri-layer barrier membrane matrix manufactured from highly purified type I collagen derived from bovine flexor tendon. The composition comprises a collagen membrane core coated on each side with a layer of bioactive glass particulate (Calcium Phosphosilicate) and bovine flexor tendon collagen as a binder

The disclosed compositions can be used for guided tissue regeneration procedures in periodontal defects to aid in wound healing post periodontal surgery.

The disclosed compositions can also be used in dental surgery procedures as a material for placement in the area of an implant, bone defect or ridge reconstruction to aid in wound healing post dental surgery.

The disclosed compositions can also be implemented in other applications where collagen matrices are utilized for tissue repair (tendon repair wraps, nerve repair conduits, etc.).

The compositions can be a variety of sizes, such as 15 mm×20 mm; 20 mm×30 mm; and 30 mm×40 mm with a thickness of 0.15 mm-0.45 mm.

The compositions can comprise: Bioglass: 1%-30%; Collagen: 70%-99%; Salts: ≤1%; and a crosslinking agent. As used herein, the term “Bioglass” or “Bioglass Particles” can be a specific example of a bioactive glass used to produce many of the products in the disclosed Examples.

Several methods can be used to produce bi and tr-layer compositions

1. Trilayer Method with Collagen/Bioglass Slurry on Top and Bottom

TABLE 1
outlines the method used to produced the compositions shown in FIG. 1
using a top-down approach - Bioglass/Collagen Slurry - Trilayer Method.

| Step | Actual | | Min | | Max | | Purpose |
|---|---|---|---|---|---|---|---|
| Shave down collagen | 2 g | | 1.5 g | | 3.5 g | | To ensure final device |
| sheets up to a final target | (0.01 | | (0.0075 | | (0.0175 | | mass is consistent |
| mass | g / cm²) | | g / cm²) | | g / cm²) | | |
| Rinse with 1X PBS | | | | | | | Neutralize pH to |
| | | | | | | | improve coating |
| | | | | | | | adherence |
| Dewater collagen sheets | | | | | | | Remove excess PBS |
| Collagen/Bioglass | | | | | | | |
| Coating (Side A): | | | | | | | |
| Dissolve NaOH in cold | 10 | mM | | | | | NaOH raises pH to |
| type 1 Water | | | | | | | improve slurry |
| | | | | | | | handling. Prevents |
| | | | | | | | collagen |
| | | | | | | | agglomeration. |
| Hydrate Bovine Tendon | 1% | (m/v) | | | | | |
| Collagen in NaOH | | | | | | | |
| solution | | | | | | | |
| Homogenize at max | 1 | min | 30 | sec | 4 | min | To blend hydrated |
| speed | | | | | | | collagen into a |
| | | | | | | | homogenous slurry |
| Mix bioglass powder into | 1% | (m/m) | 0.5% | (m/m) | 16% | (m/m) | |
| collagen slurry | | | | | | | |
| Pour Bioglass/Collagen | 0.30 | mm | 0.10 | mm | 1 | mm | |
| slurry over side A of | | | | | | | |
| dewatered collagen | | | | | | | |
| sheets. Spread evenly to | | | | | | | |
| form a coating of desired | | | | | | | |
| thickness. | | | | | | | |
| Lyophilize | | | | | | | |
| Rinse with 1X PBS | | | | | | | Neutralize pH |
| Dewater collagen sheets | | | | | | | Remove excess PBS |
| Collagen/Bioglass | | | | | | | |
| Coating (Side B): | | | | | | | |
| Dissolve NaOH in cold | 10 | mM | | | | | NaOH raises pH to |
| type 1 Water | | | | | | | improve slurry |
| | | | | | | | handling |
| Hydrate Bovine Tendon | 1% | (m/v) | | | | | |
| Collagen in NaOH | | | | | | | |
| solution | | | | | | | |
| Homogenize at max | 1 | min | 30 | sec | 4 | min | |
| speed | | | | | | | |
| Mix bioglass powder into | 1% | (m/m) | 0.5% | (m/m) | 16% | (m/m) | |
| collagen slurry | | | | | | | |
| Pour Bioglass/Collagen | 0.30 | mm | 0.10 | mm | 1 | mm | |
| slurry over side B of | | | | | | | |
| dewatered collagen | | | | | | | |
| sheets. Spread evenly to | | | | | | | |
| form a coating of desired | | | | | | | |
| thickness. | | | | | | | |
| Lyophilize | | | | | | | |
| Humidify sheets | 25% water | | 15% | | 35% | | To prepare sheets for |
| | final mass | | | | | | pressing |
| Press sheets | ≤1 | mm | | | | | To lock sheet shape |
| | | | | | | | into a thin membrane |
| Vacuum dry | | | | | | | |
| Soak sheets in | 0.05% | w/w | 0.01% | w/w | 0.1 | w/w | Collagen crosslinking |
| Glutaraldehyde solution | | | | | | | to extend in vivo |
| | | | | | | | residence time, |
| | | | | | | | improve strength, and |
| | | | | | | | improve barrier |
| | | | | | | | function |
| Rinse with type I water | | | | | | | Remove excess |
| | | | | | | | Glutaraldehyde |
| Rinse with Sodium | 1% | m/v | 0.5% | m/v | 2% | m/v | Reduce residual |

| Borohydride solution | | | | | aldehydes |
|---|---|---|---|---|---|
| Rinse with type I water | | | | | Remove excess |
| | | | | | Sodium Borohydride |
| Dewater sheets | | | | | Remove excess water |
| Vacuum dry | | | | | |
| Humidify sheets | 25% water | | 15% | 35% | To prepare sheets for |
| | final mass | | | | pressing |
| Press sheets | ≤1 | mm | | | To lock sheet shape |

| | |
|---|---|
| | into a thin membrane |
| Vacuum dry | |
| Cut sheet into desired | |
| device sizes | |
| Package device in an | |
| appropriate sterile barrier | |
| system | |
| Sterilize | |

2. Bilayer Method with Collagen/Bioglass Slurry on One Side

An alternate approach is a top-down approach—Bioglass/Collagen Slurry—bilayer Method that produces the composition of FIG. 2. This approach is the same as the method in Table 1, but where the Bioglass/Collagen slurry is poured on one side of the base collagen only. The composition of FIG. 2 consists of two layers: 1) 100% Collagen, 2) Collagen/Bioglass composite

3. Trilayer Method with Collagen/Bioglass Slurry on Top and Bottom with Bioglass on One Side of the Collagen Base

An alternate approach is a top-down approach—wound matrix like—trilayer method that produces the composition of FIG. 3. This approach is similar to method of Table 1, but where bioglass is added to the composition in a “wound matrix like” technique. A pin roller is used to perforate the base collagen layer. Bioglass is powdered on top of the perforated collagen base. The collagen base is saturated with water. A collagen slurry is dispensed on top to lock the bioglass in. The bioglass addition conducted on both sides forms a Trilayer composition.

4. Bilayer Method with Collagen/Bioglass Slurry on One Side and with Bioglass on One Side of the Collagen Base

An alternate approach is a top-down approach—wound matrix like—bilayer method that produces the composition of FIG. 4. This approach is similar to method of “3.” above, but where the bioglass is added on one side only to form a Bilayer composition. The composition consists of two layers: 1) 100% Collagen, 2) Collagen/Bioglass composite.

5. Trilayer Method with Collagen/Bioglass Slurry on Top and Bottom with a Lyophilized Collagen Slurry/Sponge

An alternate approach is a bottom-up approach—collagen base—bioglass/collagen slurry—trilayer method that produces the composition of FIG. 5. This approach has a base collagen layer prepared by producing a collagen slurry then freeze drying into a sponge, and then pressing. Then this collagen base is coated on both sides like the method of Table 1.

6. Bilayer Method with Collagen/Bioglass Slurry on One Side with a Lyophilized Collagen Slurry/Sponge

An alternate approach is a bottom-up approach—collagen base—bioglass/collagen slurry—bilayer method that produces the composition of FIG. 6. This approach is the same as the method of “5.” above, but where the collagen base is coated on one side only to form a bilayer composition.

7. Trilayer Method with Collagen/Bioglass Slurry on Top and Bottom with a Lyophilized Collagen Slurry/Sponge with Bioglass on One Side of the Collagen Slurry

An alternate approach is a bottom-up approach—collagen base—wound matrix like—trilayer method that produces the composition of FIG. 7. The base collagen layer is prepared like method “5.” above but coated like method “3.” (“wound matrix like” technique) The collagen/bioglass slurry is coated on both sides to form a Trilayer composition.

8. Bilayer Method with Collagen/Bioglass Slurry on One Side with a Lyophilized Collagen Slurry/Sponge with Bioglass on One Side of the Collagen Slurry

An alternate approach is a bottom-up approach—collagen base—wound matrix like—bilayer method that produces the composition of FIG. 8. This approach is the same as the method of “7.” above, but the collagen base is coated on one side only to form a bilayer composition.

### 9. Single Layer Method

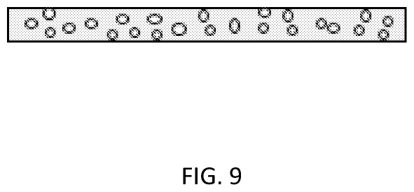

An alternate approach is a bottom-up approach—collagen/bioglass slurry—monolayer method that produces the composition of FIG. 9. In this method, the composition consists of a single layer of collagen/bioglass composite. The composition is prepared by mixing a collagen slurry with bioglass and drying altogether into a membrane composition.

Those skilled in the art will recognize, or be able to ascertain using no more than routine experimentation, many equivalents to the specific embodiments of the method and compositions described herein. Such equivalents are intended to be encompassed by the following claims.

We claim:

1. A composition comprising a tri-layered collagen composition, wherein the composition comprises
a first layer comprising a collagen:bioactive glass mixture;
a second layer comprising collagen; and
a third layer comprising a collagen:bioactive glass mixture.

2. The composition of claim 1, wherein the collagen in all three layers is derived from the same source.

3. The composition of claim 1, wherein the collagen in the first and third layers is derived from the same source.

4. The composition of any one of claims 1-3, wherein the collagen is marine, bovine, porcine, ovine, vegan, equine or recombinant collagen.

5. The composition of claim 4, wherein the collagen is purified type I collagen derived from bovine flexor tendon.

6. The composition of any one of claims 1-5, wherein the collagen in at least one layer is crosslinked.

7. The composition of any of claims 1-6, further comprising a crosslinking agent.

8. The composition of claim 7, wherein the crosslinking agent is citric acid, glycine, lysine, glutamic acid, serine, trilysine, glutaraldehyde, transglutaminase, ethylene oxide, phosphate (cross linking aid), tris, carbodiimide, heat, irradiation, EDC-NHS, ribose or other sugars, or combinations thereof.

9. The composition of claim 8, wherein the crosslinking agent is glutaraldehyde.

10. The composition of claim 9, wherein the glutaraldehyde is in a final concentration of <10%/wt.

11. The composition of any one of claims 1-10, wherein the bioactive glass is 45S5, 58S, S53P4, or a Borate-formulation.

12. The composition of any one of claims 1-11, wherein the composition is lyophilized or freeze-dried.

13. The composition of any one of claims 1-12, wherein the composition has a density of about 600 mg/cc.

14. The composition of any one of claims 1-13, wherein the composition has a pH of about 7-10.

15. The composition of any one of claims 1-14, wherein the composition has a thickness of 0.15 mm-0.45 mm.

16. The composition of any one of claims 1-15, wherein the composition comprises 1-30% bioactive glass.

17. The composition of any one of claims 1-16, wherein the composition comprises 70-99% collagen.

18. The composition of any one of claims 1-17, wherein the composition comprises 0-1% crosslinking agent

19. A method of making a tri-layered collagen composition comprising
a) preparing a collagen slurry;
b) adding bioactive glass to the collagen slurry, forming a bioactive glass collagen slurry mix;
c) coating the first side of a collagen base, having a first side and a second side, with the bioactive glass collagen slurry mix of step b), forming a single-coated collagen base;
d) drying the single-coated collagen base;
e) coating the second side of the collagen base with a bioactive glass collagen slurry mix, forming a double-coated collagen base;
f) drying the double-coated collagen base, thereby forming a dried collagen product;
g) pressing the dried collagen product, thereby forming a pressed, dried collagen product;
h) drying the pressed, lyophilized collagen product; thereby forming a dried, pressed collagen product;
i) crosslinking the dried collagen product of h) thereby forming a crosslinked collagen product;
j) drying the crosslinked collagen product of i), thereby forming a dried, crosslinked collagen product;
k) pressing the dried, crosslinked collagen product of j), thereby forming a pressed, dried, crosslinked collagen product; and
l) drying the pressed, dried, crosslinked collagen product of k), thereby forming a tri-layered collagen product.

20. The method of claim 19, wherein the collagen base is purified collagen.

21. The method of claim 20, wherein the purified collagen is purified type I collagen derived from bovine flexor tendon.

22. The method of any one of claims 19-21, wherein pressing the lyophilized double-coated collagen base locks in a shape of the lyophilized double-coated collagen base

23. The method of any one of claims 19-22, wherein pressing the lyophilized double-coated collagen base results in the lyophilized double-coated collagen base having a thickness of ≤1 mm.

24. The method of any one of claims 19-23, wherein crosslinking is a chemical crosslinking.

25. The method of claim 24, wherein the chemical crosslinking comprises contacting the dried collagen product of h) with glutaraldehyde.

26. The method of any one of claims 19-25, wherein crosslinking of step i) comprises soaking the dried collagen product of h) in a solution comprising 0.01% w/w to 0.1% w/w glutaraldehyde.

27. The method of any one of claims 19-26, further comprising a rinsing step after the crosslinking step of i) and before the drying step of j).

28. The method of claim 27, wherein rinsing comprises rinsing the dried collagen product of h) with sodium borohydride.

29. The method of claim 28, wherein the sodium borohydride neutralizes the aldehydes in the cross-liking step.

30. The method of claim 27, wherein rinsing further comprises rinsing with water before and/or after rinsing with sodium borohydride.

31. The method of any one of claims 19-30, wherein one or more of the drying steps comprises vacuum drying, freeze drying and/or air drying.

32. The method of any one of claims 19-31, further comprising cutting the final product of l).

33. The method of any one of claims 19-32, further comprising packaging the final product of l).

34. The method of any one of claims 19-33, further comprising sterilizing the final product of l).

35. The method of any one of claims 19-34, wherein the collagen slurry used to coat the first side of the collagen base is different from the collagen slurry used to coat the second side of the collagen base.

36. The method of any one of claims 19-35, wherein the drying of step d) and/or f) comprises lyophilization.

37. A method of making a bilayer collagen composition comprising
a) preparing a collagen slurry;
b) adding bioactive glass to the collagen slurry, forming a bioactive glass collagen slurry mix;
c) coating one side of a collagen base with the bioactive glass collagen slurry mix, forming a single-coated collagen base;
d) drying the single-coated collagen base, thereby forming a dried collagen product;
e) pressing the dried collagen product, thereby forming a pressed, dried collagen product;
f) drying the pressed, dried collagen product, thereby forming a dried collagen product;
g) crosslinking the dried collagen product, thereby forming a crosslinked collagen product;
h) drying the crosslinked collagen product, thereby forming a dried, crosslinked collagen product;
i) pressing the dried, crosslinked collagen product, thereby forming a pressed, dried, crosslinked collagen product
j) drying the pressed, dried, crosslinked collagen product, thereby forming a bilayered collagen product.

38. A method of making a tri-layered collagen composition comprising
a) preparing a collagen slurry;
b) drying a least a portion of the collagen slurry;
c) pressing the lyophilized collagen slurry to form a collagen base;
d) perforating the first side of a collagen base, having a first side and a second side, thereby forming a perforated collagen base;
e) adding bioactive glass to the first side of the perforated collagen base;
f) coating the first side of the perforated collagen base comprising bioactive glass with the collagen slurry, thereby forming a single-coated collagen base;
g) drying the single-coated collagen base;
h) perforating the second side of the collagen base;
i) adding bioactive glass to the second side of the perforated collagen base;
j) coating the second side of the perforated collagen base comprising bioactive glass with the collagen slurry, thereby forming a double-coated collagen base;
k) lyophilizing the double-coated collagen base, thereby forming a lyophilized collagen product.
l) pressing the lyophilized collagen product, thereby forming a pressed, lyophilized collagen product;
m) drying the pressed, lyophilized collagen product; forming a dried collagen product;
n) crosslinking the dried collagen product of k), thereby forming a crosslinked collagen product;
o) drying the crosslinked collagen product of l), thereby forming a dried, crosslinked collagen product;
p) pressing the dried, crosslinked collagen product of m), thereby forming a pressed, dried, crosslinked collagen product
q) drying the pressed, dried, crosslinked collagen product of n), thereby forming a tri-layered collagen product.

39. A method of making a bilayer collagen composition comprising
a) preparing a collagen slurry;
b) drying a least a portion of the collagen slurry;
c) pressing the lyophilized collagen slurry to form a collagen base;
d) perforating one side of a collagen base;
e) adding bioactive glass to the perforated side of the collagen base;
f) coating the perforated collagen base side comprising bioactive glass with the collagen slurry, thereby forming a single-coated collagen base;
g) drying the single-coated collagen base;
h) lyophilizing the single-coated collagen base, thereby forming a lyophilized collagen product.
i) pressing the lyophilized collagen product of f), thereby forming a pressed, lyophilized collagen product;
j) drying the pressed, lyophilized collagen product of g); forming a dried collagen product
k) crosslinking the dried collagen product of h), thereby forming a crosslinked collagen product;
l) drying the crosslinked collagen product of i), thereby forming a dried, crosslinked collagen product;
m) pressing the dried, crosslinked collagen product of j), thereby forming a pressed, dried, crosslinked collagen product;
n) drying the pressed, dried, crosslinked collagen product of k), thereby forming a tri-layered collagen product.

40. A method of making a trilayer collagen composition comprising a) preparing a collagen slurry;
b) lyophilizing at least a portion of the collagen slurry;
c) pressing the lyophilized collagen slurry, thereby forming a collagen base;
d) adding bioactive glass to a collagen slurry, thereby forming a bioactive glass collagen slurry mix;
e) coating the first side of the collagen base of c), having a first side and a second side, with the bioactive glass collagen slurry mix of d), thereby forming a single-coated collagen base;
f) drying the single-coated collagen base;
g) coating the second side of the single-coated collagen base with the bioactive glass collagen slurry mix, thereby forming a double-coated collagen base;
h) lyophilizing the double-coated collagen base of g), thereby forming a lyophilized collagen product,
i) pressing the lyophilized collagen product of h), thereby forming a pressed, lyophilized collagen product;
j) drying the pressed, lyophilized collagen product of i); thereby forming a dried collagen product;
k) crosslinking the dried collagen product of j), thereby forming a crosslinked collagen product;
l) drying the crosslinked collagen product of k), thereby forming a dried, crosslinked collagen product;
m) pressing the dried, crosslinked collagen product of l), thereby forming a pressed, dried, crosslinked collagen product; and
n) drying the pressed, dried, crosslinked collagen product of m), thereby forming a tri-layered collagen product

41. A method of making a bilayer collagen composition comprising
a) preparing a collagen slurry;
b) lyophilize at least a portion of the collagen slurry;
c) pressing the lyophilized collagen slurry to form a collagen base;
d) adding bioactive glass to a collagen slurry, forming a bioactive glass collagen slurry mix;
e) coating one side of the collagen base with the bioactive glass collagen slurry mix, thereby forming a single-coated collagen base;
f) drying the single-coated collagen base;
g) lyophilizing the single-coated collagen base, thereby forming a lyophilized collagen product.
h) pressing the lyophilized collagen product of g), thereby forming a pressed, lyophilized collagen product;
i) drying the pressed, lyophilized collagen product of h); thereby forming a dried collagen product;
j) crosslinking the dried collagen product of i), thereby forming a crosslinked collagen product;
k) drying the crosslinked collagen product of j), thereby forming a dried, crosslinked collagen product;
l) pressing the dried, crosslinked collagen product of k), thereby forming a pressed, dried, crosslinked collagen product; and
m) drying the pressed, dried, crosslinked collagen product of l), thereby forming a bilayered collagen product.

42. A method of making a (above) composition comprising
a) preparing a collagen slurry;
b) lyophilizing at least a portion of the collagen slurry;
c) pressing the lyophilized collagen slurry to form a collagen base;
d) perforating the first side of a collagen base, having a first side and a second side, thereby forming a perforated collagen base;
e) adding bioactive glass to the first side of the perforated collagen base;
f) coating the first side of the perforated collagen base comprising bioactive glass with the collagen slurry, thereby forming a single-coated collagen base;
g) drying the single-coated collagen base;
h) perforating the second side of the collagen base;
i) adding bioactive glass to the second side of the perforated collagen base;
j) coating the second side of the perforated collagen base comprising bioactive glass with the collagen slurry, thereby forming a double-coated collagen base;
k) lyophilizing the double-coated collagen base, thereby forming a lyophilized collagen product.
l) pressing the lyophilized collagen product of k), thereby forming a pressed, lyophilized collagen product;
m) drying the pressed, lyophilized collagen product of l); thereby forming a dried collagen product
n) crosslinking the dried collagen product of m),
o) drying the crosslinked collagen product of n), thereby forming a dried, crosslinked collagen product;
p) pressing the dried, crosslinked collagen product of o), thereby forming a pressed, dried, crosslinked collagen product;
q) drying the pressed, dried, crosslinked collagen product of p), thereby forming a tri-layered collagen product.

43. A method of making a bilayer collagen composition comprising
a) preparing a collagen slurry;
b) lyophilizing at least a portion of the collagen slurry;
c) pressing the lyophilized collagen slurry to form a collagen base;
d) perforating one side of a collagen base;
e) adding bioactive glass to the perforated collagen base;
f) coating the side of the perforated collagen base comprising bioactive glass with the collagen slurry, forming a single-coated collagen base;
g) drying the single-coated collagen base;
h) lyophilizing the single-coated collagen base, thereby forming a lyophilized collagen product.
i) pressing the lyophilized collagen product of h),thereby forming a pressed, lyophilized collagen product;
j) drying the pressed, lyophilized collagen product of i); thereby forming a dried collagen product
k) crosslinking the dried collagen product of j), thereby forming a crosslinked collagen product,
l) drying the crosslinked collagen product of k), thereby forming a dried, crosslinked collagen product;
m) pressing the dried, crosslinked collagen product of l), thereby forming a pressed, dried, crosslinked collagen product;
n) drying the pressed, dried, crosslinked collagen product of m), thereby forming a bilayered collagen product.

44. A method of making a monolayer collagen composition comprising
a) preparing a collagen slurry;
b) adding bioactive glass to the collagen slurry, forming a bioactive glass collagen slurry mix; and
c) optionally, drying the bioactive glass collagen slurry mix, thereby forming a dried collagen product.
